SEQUENCE LISTING (2 sequences)

SEQ ID NO: 1 - DNA, 1289 bp, Homo sapiens
aggcaccgctgctccctccagtccctccgtgcagccgatgatggccctatggtccctgctccatctcaccttcctggggttcagcattaccttgctgttggtccacgggcagggcttccaagggacagcagccatctggccatccctcttcaacgtcaacttgtccaagaaggttcaggaaagcatccagattccgaacaatgggagtgcgcccctgctcgtggatgtgcgggtgtttgtctccaacgtgtttaatgtggacatcctgcgatacacaatgtcctccatgctgctgcttaggctgtcctggctggacactcgcctggcctggaacactagtgcacacccgcggcacgccatcacgctgccctgggagtctctctggacaccaaggctcaccatcctggaggcgctctgggtggactggagggaccagagcccccaggctcgagtagaccaggacggccacgtgaagctcaacctggccctcaccacggagaccaactgcaactttgagctcctccacttcccccgggaccacagcaactgcagcctcagcttctacgctctcagcaacacggcgatggagttagagttccaggcccacgtggtgaacgagattgtgagtgtcaagagggaatacgtagtttatgatctgaagacccaagtcccaccccagcagctggtgccctgcttccaggtgacgctgaggctgaagaacacggcgctcaagtccatcatcgctctcttggtgcctgcagaggcactgctgttggctgacgtgtgcggggggttgctgcccctccgggccattgagcgcataggctacaaggtgacattgctgctgagttacctcgtcctccactcctccctggtgcaggccctgcccagctcctcctcctgcaacccactgctcatttactacttcaccatcctgctgctgctgctcttcctcagcaccatagagactgtgctgctggctgggctgctggcccggggcaaccttggggccaagagcggccccagcccagccccgagaggggaacagcgagagcacggcaacccagggcctcatcctgctgaagagccctccagaggagtaaaggggtcacagagaagctggcctgagactgctgaccgcatcttcttcctcgtgtatgtggttggggtgctgtgcacccaattcgtctttgcaggaatctggatgtgggcagcgtgcaagtctgacgcagcccctggagaggctgcaccccatggcaggcggcctagactgtaaaggggcagggcct

SEQ ID NO: 2 - protein, 411 aa, Homo sapiens
MALWSLLHLTFLGFSITLLLVHGQGFQGTAAIWPSLFNVNLSKKVQESIQIPNNGSAPLLVDVRVFVSNVFNVDILRYTMSSMLLLRLSWLDTRLAWNTSAHPRHAITLPWESLWTPRLTILEALWVDWRDQSPQARVDQDGHVKLNLALTTETNCNFELLHFPRDHSNCSLSFYALSNTAMELEFQAHVVNEIVSVKREYVVYDLKTQVPPQQLVPCFQVTLRLKNTALKSIIALLVPAEALLLADVCGGLLPLRAIERIGYKVTLLLSYLVLHSSLVQALPSSSSCNPLLIYYFTILLLLLFLSTIETVLLAGLLARGNLGAKSGPSPAPRGEQREHGNPGPHPAEEPSRGVKGSQRSWPETADRIFFLVYVVGVLCTQFVFAGIWMWAACKSDAAPGEAAPHGRRPRL

(19) United States
(12) Patent Application Publication
(10) Pub. No.: US 2004/0248245 A1
(43) Pub. Date: Dec. 9, 2004

(54) Zinc activated ion channel

(76) Inventors: Ewen F. Kirkness, Olney, MD (US); Tim G. Hales, Falls Church, VA (US); Paul A. Davies, Chestnut Hill, MA (US)

Correspondence Address: GRAY CARY WARE FREDENRICH, 1625 MASSACHUSETTS AVENUE, NW, SUITE 300, WASHINGTON, DC, 20036-2247, US

(21) Appl. No.: 10/456,511

(22) Filed: Jun. 9, 2003

(51) Int. Cl. C12Q 1/68; C07H 21/04; C07K 14/705

(52) U.S. Cl. 435/69.1; 435/320.1; 435/325; 530/350; 536/23.5; 435/6

(57) ABSTRACT

Novel zinc activated ion channel (ZAC) polypeptides, proteins and nucleic acid molecules are provided. In addition to isolated, full-length ZAC proteins, isolated ZAC fusion proteins, antigenic peptides and anti-ZAC antibodies are provided. Moreover, ZAC nucleic acid molecules, recombinant expression vectors containing a nucleic acid encoding ZAC, host cells into which the expression vectors have been introduced and nonhuman transgenic animals in which a ZAC gene has been introduced or disrupted are provided. Diagnostic, screening and therapeutic methods utilizing ZAC compositions or composition that detect, bind or modulate ZAC also are provided. Methods for identifying ZAC agonists, antagonists, inverse agonists and the like are described.

Portions of the research described herein were supported by the National Institutes of Health (NIH) Grant No. R29 NS 34702 and GM 58037.

### BACKGROUND OF THE INVENTION

Type 1 transmitter-gated ion channels comprise a family of cell surface receptors. Some of those receptors bind neurotransmitters.

Subunits of type 1 transmitter-gated ion channels generally are characterized by a signal sequence, a Cys-Cys motif, four transmembrane domains and several invariant residues that underpin the secondary structure of the subunit.

Given the role ion channels have in metabolism, and the ability to treat disease by modulating the activity of cell surface molecules, identification and characterization of ion channels can provide new compositions and methods for treating disease states that involve the activity of an ion channel. The instant invention identifies and characterizes the expression of a novel zinc activated ion channel, and provides compositions and methods for applying the discovery to the identification and treatment of related diseases.

### SUMMARY OF THE INVENTION

The instant invention relates to a newly identified zinc activated ion channel (ZAC).

In one aspect, the invention relates to isolated nucleic acids selected from the group consisting of an isolated nucleic acid which encodes a human protein of amino acids as set forth in SEQ ID NO:2, variants, mutations and fragments thereof, and an isolated nucleic acid which comprises a nucleotide sequence as set forth in SEQ ID NO:1, variants, mutations and fragments thereof. Further, the invention relates to nucleic acid hybridization probes and complementary fragments, which bind to SEQ ID NO:1 or hybridization probes, and complementary fragments which bind to nucleic acids which encode the amino acid sequence as set forth in SEQ ID NO:2. Further, the invention relates to isolated nucleic acids having about 65%-99% identity to SEQ ID NO:1, including nucleic acids having about 65%-99% identity to isolated nucleic acids encoding an amino acid sequence as set forth in SEQ ID NO:2. In a related aspect, the oligonucleotides comprise at least 8 nucleotides and methods of hybridizing are contemplated comprising the steps of contacting the complementary oligonucleotide with a nucleic acid comprising the nucleotides as set forth in SEQ ID NO:1 under conditions that permit hybridization of the complement with the nucleic acid. Further, complementary fragments may serve as anti-sense oligonucleotides for methods of inhibiting the expression of ZAC, in vivo and in vitro. Such methods may comprise the steps of providing an oligonucleotide sequence consisting of the complement of the nucleotides as set forth in SEQ ID NO:1, providing a human cell comprising an mRNA compromising the sequence of nucleotides as set forth in SEQ ID NO:1 and introducing the oligonucleotide into the cell, where the expression of ZAC is inhibited by mechanisms which include inhibition of translation, triple helix formation and/or nuclease activation leading to degradation of mRNA in the cell.

The invention also relates to isolated polypeptides selected from the group consisting of purified polypeptides of amino acid sequence as set forth in SEQ ID NO:2, variants, mutations and fragments thereof, and purified polypeptides having additional amino acid residues that provide desired functional properties to the polypeptide.

The invention further relates to the nucleic acids operably linked to expression control elements, including vectors comprising the isolated nucleic acids. The invention further relates to cultured cells transformed to comprise the nucleic acids of the invention. The invention includes methods for producing a polypeptide comprising the steps of growing transformed cells comprising the nucleic acids of the invention, permitting expression and purifying the polypeptide from the cell or medium in which a cell was cultured.

A further aspect of the invention includes an isolated antibody that binds to a polypeptide of the invention, including monoclonal and polyclonal antibodies. Further, in a related aspect, methods of producing antibodies and methods for treating ZAC related diseases with an antibody that binds to ZAC are disclosed.

An additional aspect of the invention includes methods, for diagnostic purposes, for determining the presence or absence of ZAC in a biological and/or tissue sample, or for determining the activity of a ZAC.

In another aspect of the invention, therapeutic methods are disclosed for modulating ZAC activity, including administering peptides, agonists, antagonists, inverse agonists and/or antibody to a patient in need thereof.

In another aspect of the invention, methods are disclosed for identifying modulators of ZAC comprising the steps of providing a chemical moiety, providing a cell expressing ZAC and determining whether the chemical moiety modulates the biological activity of ZAC. The chemical moieties can include, but are not limited to, peptides, antibodies, agonists, inverse agonists and antagonists.

Another aspect of the invention includes therapeutic compositions, where such compositions include nucleic acids, antibodies, polypeptides, agonists, inverse agonists and antagonists. Further, methods of the invention also include methods of treating disease states and modulating ZAC activity by administering such therapeutic compositions to a patient in need thereof.

Those and other aspects of the invention will become evident on reference to the following detailed description and the attached drawings. In addition, various references are set forth below which describe in more detail certain procedures or compositions. Each of those references hereby is incorporated herein by reference in entirety as if each were individually noted for incorporation.

### BRIEF DESCRIPTION OF THE DRAWINGS

FIG. 1 provides a nucleic acid sequence of ZAC (SEQ ID NO:1).

FIG. 2 depicts an amino acid sequence of ZAC (SEQ ID NO:2)

### DETAILED DESCRIPTION OF THE INVENTION

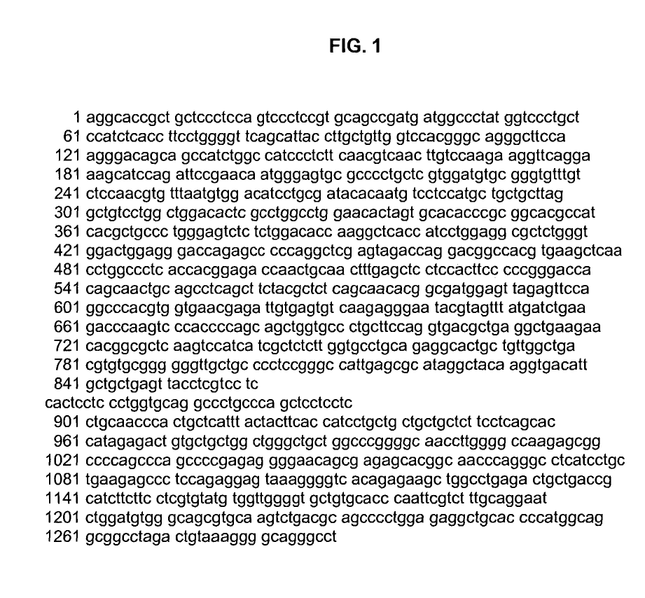

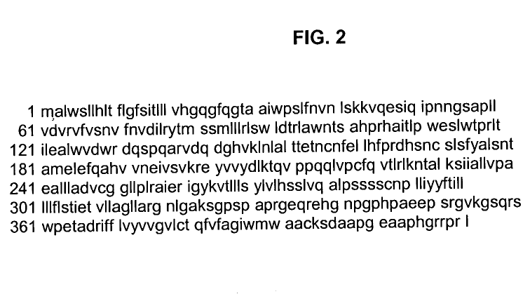

The instant invention is based on the discovery of a cDNA molecule encoding a human ZAC (hZAC), an ion channel activated by zinc. A nucleotide sequence encoding a human ZAC protein is shown in FIG. 1 (SEQ ID NO:1). An amino acid sequence of ZAC protein is shown in FIG. 2 (SEQ ID NO:2).

The ZAC cDNA of FIG. 1 (SEQ ID NO:1), which is approximately 1.27 kb in length, encodes a protein of 411 amino acids. There is a signal sequence, a Cys-Cys motif, four transmembrane domains and several invariant residues related to structure and function.

PCR and Northern blots revealed a specific mRNA fragment in prostate, thyroid, trachea, fetal whole brain, spinal cord, placenta and stomach. ZAC mRNA was not detected in adult whole brain, heart, liver, spleen or kidney cDNA.

Human ZAC is related to the type 1 transmitter-gated family of receptors having certain conserved structural and functional features. The term “family,” when referring to the protein and nucleic acid molecules of the invention, is intended to mean two or more proteins or nucleic acid molecules having an overall common structural domain. Such family members can be naturally occurring and can be from either the same or different species. For example, a family can contain a first protein of human origin and a homologue of that protein of mammalian origin, as well as a second, distinct protein of human origin and a mammalian homologue of that protein. Members of a family also may have common functional characteristics. The type 1 transmitter-gated ion channels can be classified into four structural and functional groups: those that bind nicotinic acetylcholine (nACH), γ-amino butyric acid (GABA), 5-hydroxytryptamine (5-HT) or glycine. Each of the four receptor subfamilies is composed of distinctive subunit genes that bear sequence similarity.

On the other hand, the instant ZAC has essentially no sequence identity to the known subunits, for example, having only 15% amino acid identity to the 5-HT_{3A} subunit and the α7 nACH subunit.

As used interchangeably herein, a “ZAC activity”, “biological activity of ZAC” or “functional activity of ZAC”, refers to an activity exerted by a ZAC protein, polypeptide or nucleic acid molecule in a ZAC responsive cell as determined in vivo or in vitro, according to standard techniques. A ZAC activity can be a direct activity, such as an association with or an enzymatic activity on a second protein, or an indirect activity, such as an intracellular activity mediated by interaction of the ZAC protein with a second protein, or ion flow through the ZAC protein. In a preferred embodiment, a ZAC activity includes at least one or more of the following activities: (i) the ability to bind zinc; (ii) demonstrate a transmembrane current on binding zinc; and (iii) demonstrate a reduction of transmembrane current on binding tubocuranine.

Various aspects of the invention are described in further detail in the following subsections.

### I. Isolated Nucleic Acid Molecules

One aspect of the invention pertains to isolated nucleic acid molecules that encode ZAC proteins or biologically active portions thereof; as well as nucleic acid molecules sufficient for use as hybridization probes to identify ZAC-encoding nucleic acids (e.g., ZAC mRNA) and fragments for use as PCR primers for the amplification or mutation of ZAC nucleic acid molecules. As used herein, the term “nucleic acid molecule” is intended to include DNA molecules (e.g., cDNA or genomic DNA) and RNA molecules (e.g., mRNA) as well as analogs of the DNA or RNA generated using nucleotide analogs. The nucleic acid molecule can be single-stranded or double-stranded.

An “isolated” nucleic acid molecule is one that is separated from other nucleic acid molecules that are present in the natural source of the nucleic acid. Preferably, an “isolated” nucleic acid is free of sequences (preferably protein encoding sequences) which naturally flank the nucleic acid of interest (i.e., sequences located at the 5′ and 3′ ends of the nucleic acid) in the genome of the organism from which the nucleic acid is derived. For example, in various embodiments, the isolated ZAC nucleic acid molecule can contain less than about 5 kb, 4 kb, 3 kb, 2 kb, 1 kb, 0.5 kb or 0.1 kb of nucleotide sequences that naturally flank the nucleic acid molecule in the genome of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the instant invention, e.g., a nucleic acid molecule having the nucleotide sequence of SEQ ID NO:1, or a complement of that nucleotide sequence, can be isolated using standard molecular biology techniques and the sequence information provided herein. Using all or a portion of the nucleic acid sequences of SEQ ID NO:1 as a hybridization probe, ZAC nucleic acid molecules can be isolated using standard hybridization and cloning techniques (e.g., as described in Sambrook et al., eds., “Molecular Cloning: A Laboratory Manual,” 2nd ed., Cold Spring Harbor Laboratory Press, Cold Spring Harbor, N.Y., 1989).

A nucleic acid molecule of the invention, or portion thereof, can be amplified using cDNA, mRNA or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to ZAC nucleotide sequences can be prepared by standard synthetic techniques, e.g., using an automated DNA synthesizer.

In another preferred embodiment, an isolated nucleic acid molecule of the invention comprises a nucleic acid molecule that is a complement of the nucleotide sequence shown in SEQ ID NO:1, or a portion thereof. A nucleic acid molecule which is complementary to a given nucleotide sequence is one which is sufficiently complementary to the given nucleotide sequence that it can hybridize to the given nucleotide sequence to thereby form a stable duplex.

Moreover, the nucleic acid molecule of the invention can comprise only a portion of a nucleic acid sequence encoding ZAC, for example, a fragment that can be used as a probe or primer or a fragment encoding a biologically active portion of ZAC. For example, such a fragment can comprise, but is not limited to, a region encoding nucleotides 1-21 and/or 1266-1289 as set forth in SEQ ID NO:1. The nucleotide sequence determined from cloning the human ZAC gene allows for the generation of probes and primers designed for use in identifying and/or cloning ZAC homologues in other cell types, e.g., from other tissues, as well as ZAC orthologues from other mammals. The probe/primer typically comprises substantially purified oligonucleotide. The oligonucleotide typically comprises a region of nucleotide sequence that hybridizes under stringent conditions to at least about 8-12, preferably about 25, or about 50, 75, 100, 125, 150, 175, 200, 250, 300, 350 or 400 consecutive nucleotides of the sense or anti-sense sequence of SEQ ID NO:1 or of a naturally occurring mutant of SEQ ID NO:1. Probes based on the human ZAC nucleotide sequence can be used to detect transcripts or genomic sequences encoding the similar or identical proteins. The probe may comprise a label group attached thereto, e.g., a radioisotope, a fluorescent compound, an enzyme or an enzyme co-factor. Such probes can be used as part of a diagnostic test kit for identifying cells or tissues that improperly express a ZAC protein, such as by measuring levels of a ZAC-encoding nucleic acid in a sample of cells from a subject, e.g., detecting ZAC mRNA levels or determining whether a genomic ZAC gene has been mutated or deleted.

A nucleic acid fragment encoding a “biologically active portion of ZAC” can be prepared by isolating a portion of SEQ ID NO:1 which encodes a polypeptide having a ZAC biological activity, expressing the encoded portion of ZAC protein (e.g., by recombinant expression in vitro) and assessing the activity of the encoded portion of ZAC. The invention further encompasses nucleic acid molecules that differ from the nucleotide sequence of SEQ ID NO:1 due to degeneracy of the genetic code yet encode the same ZAC protein as that encoded by the nucleotide sequence shown in SEQ ID NO:1.

In addition to the human ZAC nucleotide sequence shown in SEQ ID NO:1, it will be appreciated by those skilled in the art that DNA sequence polymorphisms that lead to changes in the amino acid sequences of ZAC may exist within a population (e.g., the human population). Such genetic polymorphism in the ZAC gene may exist among individuals within a population due to natural allelic variation. An allele is one of a group of genes that occur alternatively at a given genetic locus. As used herein, the terms “gene” and “recombinant gene” refer to nucleic acid molecules comprising an open reading frame encoding a ZAC protein, preferably a mammalian ZAC protein. As used herein, the phrase “allelic variant” refers to a nucleotide sequence that occurs at a ZAC locus or to a polypeptide encoded by the nucleotide sequence. Alternative alleles can be identified by sequencing the gene of interest in a number of different individuals. That can be carried out readily by using hybridization probes to identify the same genetic locus in a variety of individuals. Any and all such nucleotide variations and resulting amino acid polymorphisms or variations in ZAC that are the result of natural allelic variation and that do not alter the functional activity of ZAC are intended to be within the scope of the invention.

Moreover, nucleic acid molecules encoding ZAC proteins from other species (ZAC orthologues), that have a nucleotide sequence that differs from that of a human ZAC, are intended to be within the scope of the invention. Nucleic acid molecules corresponding to natural allelic variants and homologues of the ZAC cDNA as well as orthologues of the invention can be isolated based on identity to the human ZAC nucleic acids disclosed herein using the human cDNAs, or a portion thereof, as a hybridization probe according to standard hybridization techniques under stringent or at least specific hybridization conditions that enable cross hybridization.

Accordingly, in another embodiment, an isolated nucleic acid molecule of the invention is at least 300, 325, 350, 375, 400, 425, 450, 500, 550, 600, 650, 700, 800, 900, 1000 or 1100 nucleotides in length and hybridizes under stringent conditions to the nucleic acid molecule comprising the nucleotide sequence, preferably the coding sequence of SEQ ID NO:1, or a complement thereof.

As used herein, the term “hybridizes under stringent conditions” is intended to describe conditions for hybridization and washing under which nucleotide sequences at least 60% (65%, 70%, preferably 75% or greater) identical to each other typically remain hybridized to each other. Such stringent conditions are known to those skilled in the art and can be found, for example, in “Current Protocols in Molecular Biology,” John Wiley & Sons, N.Y. (1989), 6.3.1-6.3.6. A non-limiting example of stringent hybridization conditions is hybridization in 6×sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to the sequence of SEQ ID NO:1 or the complement thereof corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the ZAC sequence that may exist in the population, the skilled artisan will further appreciate that changes can be introduced by mutation into the nucleotide sequence of SEQ ID NO:1, thereby leading to changes in the amino acid sequence of the encoded ZAC protein, without substantially altering the biological activity of the ZAC protein. For example, one can make nucleotide substitutions leading to amino acid substitutions at “non-essential” amino acid residues. A “non-essential” amino acid residue is a residue that can be altered from the wild-type sequence of ZAC (e.g., the sequence of SEQ ID NO:2) without altering the biological activity, whereas an “essential” amino acid residue may be required for maintaining biological activity. For example, amino acid residues that are not conserved or only semi-conserved among ZAC of various species may be non-essential for activity and thus would be likely targets for alteration. Alternatively, amino acid residues that are conserved among the ZAC proteins of various species may be essential for activity and thus would not be likely targets for alteration.

Accordingly, another aspect of the invention pertains to nucleic acid molecules encoding ZAC proteins that contain changes in amino acid residues that are not essential for activity. Such ZAC proteins differ in amino acid sequence from SEQ ID NO:2 yet retain biological activity. In one embodiment, the isolated nucleic acid molecule includes a nucleotide sequence encoding a protein that includes an amino acid sequence that is at least about 45% identical, 65%, 75%, 85%, 95%, 98% or 99% identical to the amino acid sequence of SEQ ID NO:2.

An isolated nucleic acid molecule encoding a ZAC protein having a sequence which differs from that of SEQ ID NO:2 can be created by introducing one or more nucleotide substitutions, additions or deletions into the nucleotide sequence of SEQ ID NO:1 such that one or more amino acid substitutions, additions or deletions are introduced into the encoded protein.

Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. Those families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in ZAC is preferably replaced with another amino acid residue from the same side chain family. Alternatively, mutations can be introduced randomly along all or part of a ZAC coding sequence, such as by saturation mutagenesis, and the resultant mutants can be screened for ZAC biological activity to identify mutants that retain activity. Following mutagenesis, the encoded protein can be expressed recombinantly and the activity of the protein can be determined.

In a preferred embodiment, a mutant ZAC protein can be assayed for: (1) the ability to conduct ions in the ZAC signaling pathway; (2) the ability to bind a ZAC modulator (e.g., zinc); or (3) the ability to bind to an intracellular target protein; (4) whether activated by zinc; or (5) whether inhibited by tubocurarine.

The instant invention encompasses antisense nucleic acid molecules, i.e., molecules that are complementary to a sense nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire ZAC coding strand, or to only a portion thereof, e.g., all or part of the protein coding region (or open reading frame). An antisense nucleic acid molecule can be antisense to a noncoding region of the coding strand of a nucleotide sequence encoding ZAC. The noncoding regions (“5′ and 3′ untranslated or flanking regions”) are the 5′ and 3′ sequences that flank the coding region and are not translated into amino acids.

Given the coding strand sequences encoding ZAC disclosed herein (e.g., SEQ ID NO:1), antisense nucleic acids of the invention can be designed according to the rules of Watson and Crick base pairing. The antisense nucleic acid molecule can be complementary to the entire coding region of ZAC mRNA, but more preferably is an oligonucleotide that is antisense to only a portion of the coding or noncoding region of ZAC mRNA. For example, the antisense oligonucleotide can be complementary to the region near the translation start site of ZAC mRNA. Alternatively, the antisense molecule can be directed to regulatory regions associated with expression of ZAC. An antisense oligonucleotide can be, for example, about 5, 10, 15, 20, 25, 30, 35, 40, 45 or 50 nucleotides in length. An antisense nucleic acid of the invention can be made by cloning a suitable molecule or can be constructed using chemical synthesis and enzymatic ligation reactions using procedures known in the art. For example, an antisense nucleic acid (e.g., an antisense oligonucleotide) can be synthesized chemically using naturally occurring nucleotides or variously modified nucleotides designed to increase the biological stability of the molecules or to increase the physical stability of the duplex formed between the antisense and sense nucleic acids, e.g., phosphorothioate derivatives, phosphonate derivatives and acridine substituted nucleotides can be used.

Examples of modified nucleotides which can be used to generate the nucleic acid molecules include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, β-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-adenine, 7-methylguanine, 5-methylaminomethyluracil, 5-methoxyaminomethyl-2-thiouracil, β-D-mannosylqueosine, 5-methoxycarboxymethyluracil, 5-methoxyuracil, 2-methylthio-N⁶-isopentenyladenine, uracil-5-oxyacetic acid, butoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil and 2,6-diaminopurine. Alternatively, the nucleic acid can be produced biologically using an expression vector into which a nucleic acid has been subcloned, for example, in an antisense orientation (i.e., RNA transcribed from the inserted nucleic acid will be of an antisense orientation to a target nucleic acid of interest, described further in the following subsection).

An antisense nucleic acid molecule of the invention can be an α-anomeric nucleic acid molecule. An α-anomeric nucleic acid molecule forms specific double-stranded hybrids with complementary RNA in which the strands run parallel to each other (Gaultier et al., Nucleic Acids Res (1987)15:6625-6641). The nucleic acid molecule also can comprise a methylribonucleotide (Inoue et al., (1987) Nucleic Acids Res 15:6131-6148) or a chimeric RNA-DNA analogue (Inoue et al., (1987) FEBS Lett 215:327-330).

The invention also encompasses ribozymes. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff et al., Nature (1988) 334:585-591)) can be used to catalytically cleave ZAC mRNA transcripts to thereby inhibit translation of ZAC mRNA. A ribozyme having specificity for a ZAC-encoding nucleic acid can be designed based on the nucleotide sequence of a ZAC cDNA disclosed herein (e.g., SEQ ID NO:1). For example, a derivative of a Tetrahymena L-19 IVS RNA can be constructed to contain to the nucleotide sequence to cleave a ZAC-encoding mRNA, see, e.g., Cech et al., U.S. Pat. No. 4,987,071; and Cech et al., U.S. Pat. No. 5,116,742. Alternatively, ZAC mRNA can be used to select a catalytic RNA having a specific ribonuclease activity from a pool of RNA molecules, see, e.g., Bartel et al., Science (1993) 261:1411-1418.

The invention also encompasses nucleic acid molecules that form triple helical structures. For example, ZAC gene expression can be inhibited by targeting nucleotide sequences complementary to the regulatory region of the ZAC (e.g., the ZAC promoter and/or enhancers) to form triple helical structures that prevent transcription of the ZAC gene in target cells, see generally Helene, Anticancer Drug Dis (1991) 6(6):569; Helene, Ann NY Acad Sci (1992) 660:27; and Maher, Bioassays (1992) 14(12):807.

In preferred embodiments, the nucleic acid molecules of the invention can be modified at the base moiety, sugar moiety or phosphate backbone to improve, e.g., the stability, hybridization or solubility of the molecule. For example, the deoxyribose phosphate backbone of the nucleic acids can be modified to generate peptide nucleic acids (see Hyrup et al., Bioorganic & Medicinal Chemistry (1996) 4:5). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) supra; Perry-OKeefe et al., Proc Natl Acad Sci USA (1996) 93:14670.

PNAs of ZAC can be used in therapeutic and diagnostic applications. For example, PNAs can be used as antisense or antigene agents for sequence-specific modulation of gene expression by, e.g., inducing transcription or translation arrest or inhibiting replication. PNAs of ZAC can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra) or as probes or primers for DNA sequence and hybridization (Hyrup (1996) supra; Perry-O'Keefe et al. (1996) supra).

In another embodiment, PNAs of ZAC can be modified, e.g., to enhance their stability, specificity or cellular uptake, by attaching lipophilic or other helper groups to PNA, by the formation of PNA-DNA chimeras, or by the use of liposomes or other techniques of drug delivery known in the art. The synthesis of PNA-DNA chimeras can be performed as described in Hyrup (1996) supra Finn et al., Nucleic Acids Res (1996) 24(17):3357-63, Mag et al., Nucleic Acids Res (1989) 17:5973; and Peterser et al., Bioorganic Med Chem Lett (1975) 5:1119.

### II. Isolated ZAC Proteins and Anti-ZAC Antibodies

One aspect of the invention pertains to isolated ZAC proteins, and biologically active portions thereof, as well as polypeptide fraginents suitable, for example, for use as immunogens to raise anti-ZAC antibodies. In one embodiment, native ZAC proteins can be isolated from cells or tissue sources by an appropriate purification scheme using standard protein purification techniques. In another embodiment, ZAC proteins are produced by recombinant DNA techniques. Alternative to recombinant expression, a ZAC protein or polypeptide can be synthesized chemically using standard peptide synthesis techniques.

An “isolated” or “purified” protein or biologically active portion thereof is substantially free of cellular material or other contaminating proteins from the cell or tissue source from which the ZAC protein is derived, or substantially free of chemical precursors or other chemicals when chemically synthesized. The language “substantially free of cellular material” includes preparations of ZAC protein wherein the protein is separated from cellular components of the cells from which it is isolated or recombinantly produced. Thus, ZAC protein that is substantially free of cellular material includes preparations of ZAC protein having less than about 30%, 20%, 10% or 5% (by dry weight) of non-ZAC protein (also referred to herein as a “contaminating protein”). When the ZAC protein or biologically active portion thereof is produced recombinantly, it is also preferably substantially free of culture medium, i.e., culture medium represents less than about 20%, 10% or 5% of the volume of the protein preparation. When ZAC protein is produced by chemical synthesis, it is preferably substantially free of chemical precursors or other chemicals, i.e., it is separated from chemical precursors or other chemicals that are involved in the synthesis of the protein. Accordingly, such preparations of ZAC protein have less than about 30%, 20%, 10% or 5% (by dry weight) of chemical precursors or non-ZAC chemicals.

Biologically active portions of a ZAC protein include peptides comprising amino acid sequences sufficiently identical to or derived from the amino acid sequence of the ZAC protein (e.g., the amino acid sequence shown in SEQ ID NO:2), which include fewer amino acids than the full length ZAC proteins, and exhibit at least one activity of a ZAC protein. Typically, biologically active portions comprise a domain or motif with at least one activity of the ZAC protein. A biologically active portion of a ZAC protein can be a polypeptide that is, for example, 10, 25, 50, 100 or more amino acids in length.

Moreover, other biologically active portions, in which other regions of the protein are deleted, can be prepared by recombinant techniques and evaluated for one or more of the functional activities of a native ZAC protein.

A preferred ZAC protein has the amino acid sequence of SEQ ID NO:2. Other useful ZAC proteins are substantially identical to SEQ ID NO:2 and retain the functional activity of the protein of SEQ ID NO:2 yet differ in amino acid sequence due to natural allelic variation or mutagenesis. For example, such ZAC proteins and polypeptides possess at least one biological activity described herein.

Accordingly, a useful ZAC protein is a protein that includes an amino acid sequence at least about 45%, preferably 55%, 65%, 75%, 85%, 95% or 99% identical to the amino acid sequence of SEQ ID NO:2 and retains the functional activity of the ZAC proteins of SEQ ID NO:2.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions then are compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., percent identity=number of identical positions/total number of positions (e.g., overlapping positions) ×100). In one embodiment, the two sequences are the same length.

The determination of percent identity between two sequences can be accomplished using a mathematical algorithm. A preferred, non-limiting example of a mathematical algorithm utilized for the comparison of two sequences is the algorithm of Karlin et al., Proc Natl Acad Sci USA (1990) 87:2264, modified as in Karlin et al., Proc Natl Acad Sci USA (1993) 90:5873-5877. Such an algorithm is incorporated into the NBLAST and XBLAST programs of Altschul et al., J Mol Bio (1990) 215:403. BLAST nucleotide searches can be performed with the NBLAST program, score=100, wordlength=12 to obtain nucleotide sequences homologous to ZAC nucleic acid molecules of the invention. BLAST protein searches can be performed with the XBLAST program, score=50, wordlength=3, to obtain amino acid sequences homologous to ZAC protein molecules of the invention. To obtain gapped aligrrnents for comparison purposes, Gapped BLAST can be utilized as described in Altschul et al., Nucleic Acids Res (1997) 25:3389. Alternatively, PSI-Blast can be used to perform an iterated search that detects distant relationships between molecules, Altschul et al. (1997) supra. When utilizing BLAST, Gapped BLAST, and PSI-Blast programs, the default parameters of the respective programs (e.g., XBLAST and NBLAST) can be used, see http://www.ncbi.nlm.nih.gov.

Another preferred, non-limiting example of a mathematical algorithm utilized for the comparison of sequences is the algorithm of Myers et al., CABIOS (1988) 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The invention also provides ZAC chimeric or fusion proteins. As used herein, a ZAC “chimeric protein” or “fusion protein” comprises a ZAC polypeptide operably linked to a non-ZAC polypeptide. A “ZAC polypeptide” refers to a polypeptide having an amino acid sequence corresponding to ZAC, whereas a “non-ZAC polypeptide” refers to a polypeptide having an amino acid sequence corresponding to a protein which is not substantially identical to the ZAC protein, e.g., a protein which is different from the ZAC protein. The non-ZAC polypeptide may originate from the same or from a different organism. Within a ZAC fusion protein the ZAC polypeptide can correspond to all or a portion of a ZAC protein, preferably at least one biologically active portion of a ZAC protein. Within the fusion protein, the term “operably linked” is intended to indicate that the ZAC polypeptide and the non-ZAC polypeptide are fused in-frame to each other. The non-ZAC polypeptide can be fused to the N-terminus or C-terminus of the ZAC polypeptide. One useful fusion protein is a GST-ZAC fusion protein in which the ZAC sequences are fused to the C-terminus of a glutathione-S-transferase (GST) sequence. Such fusion proteins can facilitate the purification of recombinant ZAC, which can be cloned into a vector, such as pGEX-2T. The resulting construct can be introduced into a host cell (e.g., E. coli) and expression from said construct can be induced by an appropriate small molecule (e.g., isopropyl-1-thio-β-D-galactopyranoside) and subsequently purified (see, e.g., Lee et al., J Biol Chem (1996) 271(19):11272-11279).

In yet another embodiment, the fusion protein is a ZAC-immunoglobulin fusion protein in which all or part of ZAC is fused to sequences derived from a member of the immunoglobulin protein family. The ZAC-immunoglobulin fusion proteins of the invention can be incorporated into pharmaceutical compositions and administered to a subject to inhibit an interaction between a ZAC ligand or modulator and a ZAC protein on the surface of a cell, to thereby suppress ZAC-mediated cellular activity. The ZAC-immunoglobulin fusion protein can be used to affect the bioavailability of a ZAC cognate ligand or modulator. Inhibition of the interaction may be useful therapeutically, both for treating proliferative and differentiative disorders and for modulating (e.g. promoting or inhibiting) cell survival. Moreover, the ZAC-immunoglobulin fusion proteins of the invention can be used as immunogens to produce anti-ZAC antibodies in a subject, to purify ZAC ligands and in screening assays to identify molecules that inhibit the interaction of ZAC with a ZAC ligand.

Preferably, a ZAC chimeric or fusion protein of the invention is produced by standard recombinant DNA techniques. For example, DNA fragments coding for the different polypeptide sequences are ligated together in-frame in accordance with conventional techniques, for example, by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which subsequently can be annealed and reamplified to generate a chimeric gene sequence (see e.g., Ausubel et al., supra). Moreover, many expression vectors are commercially available that already encode a fusion moiety (e.g., a GST polypeptide). A ZAC-encoding nucleic acid can be cloned into such an expression vector such that the fusion moiety is linked in-frame to the ZAC protein.

The instant invention also pertains to variants of the ZAC protein (i.e., proteins having a sequence that differs from that of the ZAC amino acid sequence). Such variants can function as either ZAC agonists (mimetics) or as ZAC antagonists. Variants of the ZAC protein can be generated by mutagenesis, e.g., discrete point mutation or truncation of the ZAC protein. An agonist of the ZAC protein can retain substantially the same, or a subset, of the biological activities of the naturally occurring form of the ZAC protein. An antagonist of the ZAC protein can inhibit one or more of the activities of the naturally occurring form of the ZAC protein by, for example, competitively binding to a downstream or upstream member of a cellular signaling cascade that includes the ZAC protein. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the ZAC proteins.

Variants of the ZAC protein which function as either ZAC agonists (mimetics) or more likely as ZAC antagonists can be identified by screening combinatorial libraries of mutants, e.g., truncation mutants, of the ZAC protein for ZAC protein agonist or antagonist activity. In one embodiment, a variegated library of ZAC variants is generated by combinatorial mutagenesis at the nucleic acid level and is encoded by a variegated gene library. A variegated library of ZAC variants can be produced by, for example, enzymatically ligating a mixture of synthetic oligonucleotides into gene sequences such that a degenerate set of potential ZAC sequences is expressed as individual polypeptides, or alternatively, as a set of larger fusion proteins (e.g., for phage display) containing the set of ZAC sequences therein. There are a variety of methods that can be used to produce libraries of potential ZAC variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential ZAC sequences. Methods for synthesizing degenerate oligonucleotides are known in the art (see, e.g., Narang, Tetrahedron (1983) 39:3; Itakura et al., Ann Rev Biochem (1984) 53:323; Itakura et al., Science (1984) 198:1056; Ike et al., Nucleic Acid Res (1983) 11:477).

In addition, libraries of fragments of the ZAC protein coding sequence can be used to generate a variegated population of ZAC fragments for screening and subsequent selection of variants of a ZAC protein. In one embodiment, a library of coding sequence fragments can be generated by treating a double-stranded PCR fragment of a ZAC coding sequence with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with S1 nuclease, and ligating the resulting fragment library into an expression vector. By that method, an expression library can be derived which encodes N-terninal and internal fragments of various sizes of the ZAC protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of ZAC proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify ZAC variants (Arkin et al., Proc Natl Acad Sci USA (1992) 89:7811-7815; Delgrave et al., Protein Engineering (1993) 6(3):327-331).

An isolated ZAC protein, or a portion or fragment thereof, can be used as an immunogen to generate antibodies that bind ZAC using standard techniques for polyclonal and monoclonal antibody preparation. The full-length ZAC protein can be used or, alternatively, the invention provides antigenic peptide fragments of ZAC for use as immunogens. The antigenic peptide of ZAC comprises at least 8 (preferably 10, 15, 20 or 30) amino acid residues of the amino acid sequence shown in SEQ ID NO:2 and encompasses an epitope of ZAC such that an antibody raised against the peptide forms a specific immune complex with ZAC.

In a related aspect, epitopes encompassed by the antigenic peptide are regions of ZAC that are located on the surface of the protein, e.g., hydrophilic regions, on the surface of the cell, which are distinctive from the transmembrane domains.

Another aspect of the invention pertains to anti-ZAC antibodies. The term “antibody” as used herein refers to immunoglobulin molecules and immunologically active portions of immunoglobulin molecules, i.e., molecules that contain an antigen-binding site that specifically binds ZAC. A molecule that specifically binds to ZAC is a molecule that binds ZAC, but does not substantially bind other molecules in a sample, e.g., a biological sample, which naturally contains ZAC. Examples of immunologically active portions of immunoglobulin molecules include F_{ab} and F_{(ab′)2} fragments which can be generated by treating the antibody with an enzyme such as pepsin. The invention provides polyclonal and monoclonal antibodies that bind ZAC. The term “monoclonal antibody” or “monoclonal antibody composition”, as used herein, refers to a population of antibody molecules that contain only one species of an antigen binding site capable of immunoreacting with a particular epitope of ZAC. A monoclonal antibody composition thus typically displays a single binding affinity for a particular ZAC protein with which it immunoreacts.

A ZAC immunogen typically is used to prepare antibodies by immunizing a suitable subject (e.g., rabbit, goat, mouse or other mammal) with the imunogen. An appropriate immunogenic preparation can contain, for example, recombinantly expressed ZAC protein or a chemically synthesized ZAC polypeptide. The preparation further can include an adjuvant, such as Freund's complete or incomplete adjuvant, or similar immunostimulatory agent. Immunization of a suitable subject with an immunogenic ZAC preparation induces a polyclonal anti-ZAC antibody response. The anti-ZAC antibody titer in the immunized subject can be monitored over time by standard techniques, such as with an enzyme linked immunosorbent assay (ELISA) using immobilized ZAC.

If desired, the antibody molecules directed against ZAC can be isolated from the mammal (e.g., from the blood) and further purified by well-known techniques, such as protein A chromatography to obtain the IgG fraction.

At an appropriate time after immunization, e.g., when the anti-ZAC antibody titers are highest, antibody-producing cells can be obtained from the subject and used to prepare monoclonal antibodies by standard techniques, such as the hybridoma technique originally described by Kohler et al., Nature (1975) 256:495-497, the human B cell hybridoma technique (Kozbor et al., Immunol Today (1983) 4:72), the EBV-hybridoma technique (Cole et al., Monoclonal Antibodies and Cancer Therapy, (1985), Alan R. Liss, Inc., pp. 77-96) or trioma techniques. The technology for producing hybridomas is well known (see generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with a ZAC immunogen as described above, and the culture supernatants of the resulting hybridoma cells are screened to identify a hybridoma producing a monoclonal antibody that binds ZAC.

Any of the many well known protocols used for fusing lymphocytes and immortalized cell lines can be applied for the purpose of generating an anti-ZAC monoclonal antibody (see, e.g., Current Protocols in Immunology, supra; Galfre et al., Nature (1977) 266:55052; Kenneth, in Monoclonal Antibodies: A New Dimension In Biological Analyses, Plenum Publishing Corp., New York, N.Y. (1980); and Lerner, Yale J Biol Med (1981) 54:387-402). Moreover, the ordinarily skilled worker will appreciate that there are many variations of such methods that also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the instant invention with an immortalized mouse cell line, e.g., a myeloma cell line that is sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. Those myeloma lines are available from the ATCC. Typically, HAT-sensitive mouse myeloma cells are fused to mouse splenocytes using polyethylene glycol (“PEG”). Hybridoma cells resulting from the fusion then are selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind ZAC, e.g., using a standard ELISA assay.

Alternative to preparing monoclonal antibody-secreting hybridomas, a monoclonal anti-ZAC antibody can be identified and isolated by screening a recombinant combinatorial immunoglobulin library (e.g., an antibody phage display library) with ZAC to thereby isolate immunoglobulin library members that bind ZAC. Kits for generating and screening phage display libraries are commercially available (e.g., the Pharmacia Recombinant Phage Antibody System, Catalog No. 27-9400-01; and the Stratagene SurfZAP®Phage Display Kit, Catalog No. 240612).

Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology (1991) 9:1370-1372; Hay et al., Hum Antibod Hybridomas (1992) 3:81-85; Huse et al., Science (1989) 246:1275-1281; and Griffiths et al., EMBO J (1993) 25 12:725-734.

Moreover, recombinant anti-ZAC antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; Europe Patent Application 184,187; Europe Patent Application No. 171,496; Europe Patent Application No. 173,494; PCT Publication No. WO 86/01533; U.S. Pat. No. 4,816,567; Europe Patent Application No. 125,023; Better et al ., Science (1988) 240:1041-1043; Liu et al., Proc Natl Acad Sci USA (1987) 84:3439-3443; Lin et al., J Immunol (1987) 139:3521-3526; Sun et al., Proc Natl Acad Sci USA (1987) 84:214-218; Nishimura et al., Canc Res (1987) 47:999-1005; Wood et al., Nature (1985) 314:446-449; Shaw et al., J Natl Cancer Inst (1988) 80:1553-1559; Morrison, Science (1985) 229:1202-1207; Oi et al., Bio/Techniques (1986) 4:214; U.S. Pat. No. 5,225,539; Jones et al., Nature (1986) 321:552-525; Verhoeyan et al., Science (1988) 239:1534; and Beidler et al., J Immunol (1988) 141:4053-4060.

Completely human antibodies are particularly desirable for therapeutic treatment of human patients. Such antibodies can be produced using transgenic mice that are incapable of expressing endogenous immunoglobulin heavy and light chain genes, but which can express human heavy and light chain genes. The transgenic mice are immunized in the normal fashion with a selected antigen, e.g., all or a portion of ZAC. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, an antibody that inhibits ZAC activity, is identified. The heavy chain and the light chain of the non-human antibody are cloned and used to create phage display F_{ab} fragments. For example, the heavy chain gene can be cloned into a plasmid vector so that the heavy chain can be secreted from bacteria. The light chain gene can be cloned into a phage coat protein gene so that the light chain can be expressed on the surface of phage. A repertoire (random collection) of human light chains fused to phage is used to infect the bacteria that express the non-human heavy chain. The resulting progeny phage display hybrid antibodies (human light chain/non-human heavy chain). The selected antigen is used in a panning screen to select phage which bind the selected antigen. Several rounds of selection may be required to identify such phage. Next, human light chain genes are isolated from the selected phage which bind the selected antigen. The selected human light chain genes then are used to guide the selection of human heavy chain genes. The selected human light chain genes are inserted into vectors for expression by bacteria. Bacteria expressing the selected human light chains are infected with a repertoire of human heavy chains fused to phage. The resulting progeny phage display human antibodies (human light chain/human heavy chain).

Next, the selected antigen is used in a panning screen to select phage that bind the selected antigen. The phage selected in that step display a completely human antibody that recognizes the same epitope recognized by the original selected, non-human monoclonal antibody. The genes encoding both the heavy and light chains are isolated readily and can be manipulated further for production of human antibody. The technology is described by Jespers et al. (Bio/Technology (1994) 12:899-903).

An anti-ZAC antibody (e.g., monoclonal antibody) can be used to isolate ZAC by standard techniques, such as affinity chromatography or immunoprecipitation. An anti-ZAC antibody can facilitate the purification of natural ZAC from cells and of recombinantly produced ZAC expressed in host cells. Moreover, an anti-ZAC antibody can be used to detect ZAC protein (e.g., in a cellular lysate or cell supernatant) to evaluate the abundance and pattern of expression of the ZAC protein. Anti-ZAC antibodies can be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials and radioactive materials. Examples of suitable enzymes include horseradish peroxidase, alkaline phosphatase, galactosidase or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, green fluorescent protein or phycoerythrin; an example of a luminescent material includes luminol; examples of bioluminescent materials include luciferase, luciferin or aequorin, and examples of suitable radioactive materials include ¹²⁵I, ¹³¹I, ³⁵S or ³H.

### III. Recombinant Expression Vectors and Host Cells

Another aspect of the invention pertains to vectors, preferably expression vectors, containing a nucleic acid encoding ZAC (or a portion thereof). As used herein, the term “vector” refers to a nucleic acid molecule capable of transporting another nucleic acid to which it has been linked. One type of vector is a “plasmid”, which refers to a circular double-stranded DNA loop into which additional DNA segments can be ligated. Another type of vector is a viral vector, wherein additional DNA segments can be ligated into a viral genome. Certain vectors are capable of autonomous replication in a host cell into which they are introduced (e.g., bacterial vectors having a bacterial origin of replication, and episomal mammalian vectors). Other vectors (e.g., non-episomal mammalian vectors) are integrated into the genome of a host cell on introduction into the host cell, and thereby are replicated along with the host genome. Moreover, certain vectors, expression vectors, are capable of directing the expression of genes which are operably linked therein. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), that serve equivalent functions.

The recombinant expression vectors of the invention comprise nucleic acid of the invention in a form suitable for expression of the nucleic acid in a host cell. That means that the recombinant expression vectors include one or more regulatory sequences, selected on the basis of host cells to be used for expression, which is operably linked to the nucleic acid to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell). The term “regulatory sequence” is intended to include promoters, enhancers and other expression control elements (e.g., polyadenylation signals). Such regulatory sequences are described, for example, in Goeddel, Gene Expression Technology in Methods in Enzymology Vol. 185, Academic Press, San Diego, Calif. (1990). Regulatory sequences include those that direct constitutive expression of the nucleotide sequence in many types of host cells (e.g., tissue specific regulatory sequences). It will be appreciated by those skilled in the art that the design of the expression vector can depend on such factors as the choice of host cell to be transformed, the level of expressed protein desired etc. The expression vectors of the invention can be introduced into host cells to thereby produce proteins or peptides, encoded by nucleic acids as described herein (e.g., ZAC proteins, mutant forms of ZAC, fusion proteins etc.).

The recombinant expression vectors of the invention can be designed for expression of ZAC in prokaryotic or eukaryotic cells, e.g., bacterial cells such as E. coli, insect cells (using baculovirus expression vectors), yeast cells or mammalian cells. Suitable host cells are discussed further in Goeddel, supra. Alternatively, the recombinant expression vector can be transcribed and translated in vitro, for example using T7 promoter regulatory sequences and T7 polymerase.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but still are included within the scope of the term as used herein.

A host cell can be any prokaryotic or eukaryotic cell. For example, ZAC protein can be expressed in bacterial cells such as E. coli, insect cells, yeast or mammalian cells (such as Chinese hamster ovary cells (CHO) or COS cells). Other suitable host cells are known to those skilled in the art. Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection or electroporation.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith et al., Gene (1988) 67:31-40), pMAL (New England Biolabs, Beverly, Mass.) and pRITS (Pharmacia, Piscataway, N.J.) which fuse glutathione 5-transferase (GST), maltose E binding protein or protein A, respectively, to the target recombinant protein.

Examples of suitable inducible non-fusion E. coli expression vectors include pTrc (Amann et al., Gene (1988) 69:301-315) and pET 11d (Studier et al., Gene Expression Technology in Methods in Enzymology, Academic Press, San Diego, Calif. (1990) 185:60-89). Target gene expression from the pTrc vector relies on host RNA polymerase transcription from a hybrid trp-lac fusion promoter. Target gene expression from the pET 11d vector relies on transcription from a T7 gn1-lac fusion promoter mediated by a coexpressed viral RNA polymerase (T7 gn1). The viral polymerase is supplied by host strains BL21 (DE3) or HMS 174 (DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology in Methods in Enzymology, Academic Press, San Diego, Calif. (1990) 185:119-128). Another strategy is to alter the nucleic acid sequence of the nucleic acid to be inserted into an expression vector so that the individual codons for each amino acid are those preferentially utilized in E. coli (Wada et al., Nucleic Acids Res (1992) 20:2111-2118). Such alteration of nucleic acid sequences of the invention can be carried out by standard DNA synthesis techniques.

In another embodiment, the ZAC expression vector is a expression vector. Examples of vectors for expression in S. cerevisiae include pYepSec1 (Baldari et al., EMBO J (1987) 6:229-234), pMFa (Kuijan et al., Cell (1982) 30:933-943), pJRY88 (Schultz et al., Gene (1987) 54:113-123), pYES2 (Invitrogen Corporation, San Diego, Calif.) and pPicZ (Invitrogen Corp, San Diego, Calif.).

Alternatively, ZAC can be expressed in insect cells using baculovirus expression vectors. Baculovirus vectors available for expression of proteins in cultured insect cells (e.g., Sf9 cells) include the pAc series (Smith et al., Mol Cell Biol (1983) 3:2156-2165) and the pVL series (Lucklow et al., Virology (1989) 170:31-39).

In yet another embodiment, a nucleic acid of the invention is expressed in mammalian cells using a mammalian expression vector. Examples of mammalian expression vectors include pCDM8 (Seed, Nature (1987) 329:840) and pMT2PC (Kaufman et al., EMBO J (1987) 6:187-195). When used in mammalian cells, the control functions of the expression vector often are provided by viral regulatory elements. For example, commonly used promoters are derived from polyoma, adenovirus 2, cytomegalovirus and simian virus 40. For other suitable expression systems for both prokaryotic and eukaryotic cells, see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., Genes Dev (1987) 1:268-277), lymphoid-specific promoters (Calame et al., Adv Immunol (1988) 43:235-275), in particular, promoters of T cell receptors (Winoto et al., EMBO J (1989) 8:729-733) and immunoglobulins (Baneiji et al., Cell (1983) 33:729-740; Queen et al., Cell (1983) 33:741-748), neuron-specific promoters (e.g., the neurofilament promoter; Byrne et al., Proc Natl Acad Sci USA (1989) 86:5473-5477), pancreas-specific promoters (Edlund et al., Science (1985) 230:912-916) and mammary gland-specific promoters (e.g., milk whey promoter; U.S. Pat. No. 4,873,316 and Europe Patent Application No. 264,166). Developmentally-regulated promoters also are encompassed, for example, the murine hox promoters (Kessel et al., Science (1990) 249:374-379) and the α-fetoprotein promoter (Campes et al., Genes Dev (1989) 3:537-546).

In certain host cells (e.g., mammalian host cells), expression and/or secretion of ZAC can be increased through use of a heterologous signal sequence. For example, the gp6® secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule that is antisense to ZAC mRNA. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue-specific or cell type-specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes, see Weintraub et al. (Reviews—Trends in Genetics, Vol. 1(1)1986).

For stable transformation of mammalian cells, it is known that, depending on the expression vector and transfection technique used, only a small fraction of cells may integrate the foreign DNA into the genome. To identify and to select those integrants, a gene that encodes a selectable marker (e.g., for resistance to antibiotics) generally is introduced into the host cells along with the gene of interest. Preferred selectable markers include those that confer resistance to drugs, such as G418, hygromycin and methotrexate. Nucleic acid encoding a selectable marker can be introduced into a host cell on the same vector as that encoding ZAC or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) ZAC protein. Accordingly, the invention further provides methods for producing ZAC protein using the host cells of the invention. In one embodiment, the method comprises culturing the host cell of invention (into which a recombinant expression vector encoding ZAC has been introduced) in a suitable medium such that ZAC protein is produced. In another embodiment, the method further comprises isolating ZAC from the medium or the host cell.

The host cells of the invention also can be used to produce nonhuman transgenic animals. For example, in one embodiment, a host cell of the invention is a fertilized oocyte or an embryonic stem cell into which ZAC-coding sequences have been introduced or in which the endogenous ZAC genes have been inactivated. Such host cells then can be used to create non-human transgenic animals in which exogenous ZAC sequences have been introduced into the genome or homologous recombinant animals in which endogenous ZAC sequences have been altered. Such animals are useful for studying the function and/or activity of ZAC and for identifying and/or evaluating modulators of ZAC activity. As used herein, a “transgenic animal” preferably is a mammal in which one or more of the cells of the animal include a transgene. Examples of transgenic animals include non-human primates, sheep, dogs, cows, goats, chickens, amphibians etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” preferably is a mammal, in which an endogenous ZAC gene has been altered by homologous recombination between the endogenous gene and an exogenous DNA molecule introduced into a cell of the animal, e.g., an embryonic cell of the animal, prior to development of the animal.

A transgenic animal of the invention can be created by introducing ZAC-encoding nucleic acid into the male pronuclei of a fertilized oocyte, e.g., by microinjection, and allowing the oocyte to develop in a pseudopregnant female foster animal. The ZAC cDNA sequence, e.g., that of SEQ ID NO:1, can be introduced as a transgene into the genome of a non-human animal. Alternatively, a nonhuman homologue of the human ZAC gene can be isolated based on hybridization to the human ZAC cDNA and used as a transgene. Intronic sequences and polyadenylation signals also can be included in the transgene to increase the efficiency of expression of the transgene. A tissue-specific regulatory sequence(s) can be operably linked to the ZAC transgene to direct expression of ZAC protein to particular cells. Methods for generating transgenic animals via embryo manipulation and microinjection are conventional in the art and are described, for example, in U.S. Pat. Nos. 4,736,866 and 4,870,009 and in U.S. Pat. No. 4,873,191. Similar methods are used for production of other transgenic animals. A transgenic founder animal then can be used to breed additional animals carrying the transgene. Moreover, transgenic animals carrying a transgene encoding ZAC further can be bred to other transgenic animals carrying other transgenes.

To create a homologous recombinant animal, a vector is prepared which contains at least a portion of a ZAC gene (e.g., a human or a non-human homolog of the ZAC gene) into which a deletion, addition or substitution has been introduced to thereby alter, e.g., functionally disrupt, the ZAC gene. In a preferred embodiment, the vector is designed such that, on homologous recombination, the endogenous ZAC gene is functionally disrupted (i.e., no longer encodes a functional protein; also referred to as a “knock out” animal). Alternatively, the vector can be designed such that, on homologous recombination, the endogenous ZAC gene is mutated or otherwise altered but still encodes functional protein (e.g., the upstream regulatory region can be altered to thereby alter the expression of the endogenous ZAC protein). In the homologous recombination vector, the altered portion of the ZAC gene is flanked at the 5′ and 3′ ends by additional nucleic acid of the ZAC gene to allow for homologous recombination to occur between the exogenous ZAC gene carried by the vector and an endogenous ZAC gene in an embryonic stem cell. The additional flanking ZAC nucleic acid is of sufficient length for successful homologous recombination with the endogenous gene. Typically, several kilobases of flanking DNA (both at the 5′ and 3′ ends) are included in the vector (see, e.g., Thomas et al., Cell (1987) 51:503 for a description of homologous recombination vectors). The vector is introduced into an embryonic stem cell line (e.g., by electroporation) and cells in which the introduced ZAC gene has homologously recombined with the endogenous ZAC gene are selected (see, e.g., Li et al., Cell (1992) 69:915). The selected cells then are injected into a blastocyst of an animal (e.g., a mouse) to form aggregation chimeras (see, e.g., Bradley in Teratocarcinomas and Embryonic Stem Cells: A Practical Approach, Robertson, ed., IRL, Oxford (1987) pp. 113-152). A chimeric embryo then can be implanted into a suitable pseudopregnant female foster animal and the embryo brought to term. Progeny harboring the homologously recombined DNA in germ cells can be used to breed animals in which all cells of the animal contain the homologously recombined DNA by germline transmission of the transgene. Methods for constructing homologous recombination vectors and homologous recombinant animals are described further in Bradley, Current Opinion in Bio/Technology (1991) 2:823-829 and in PCT Publication Nos. WO 90/11354, WO 91/01140, WO 92/0968 and WO 93/04169.

In another embodiment, transgenic non-human animals can be produced which contain selected systems that allow for regulated expression of the transgene. One example of such a system is the cre/loxP recombinase system of bacteriophage P1. For a description of the cre/loxP recombinase system, see, e.g., Lakso et al., Proc Natl Acad Sci USA (1992) 89:6232-6236. Another example of a recombinase system is the FLP recombinase system of S. cerevisiae (O'Gorrnan et al., Science (1991) 251:1351-1355). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein also can be produced according to the methods described in Wilmut et al., Nature (1997) 385:810-813 and PCT Publication Nos. WO 97/07668 and WO 97/07669. In brief, a cell, e.g., a somatic cell, from the transgenic animal can be isolated and induced to exit the growth cycle and enter G₀ phase. The quiescent cell then can be fused, e.g., through the use of electrical pulses, to an enucleated oocyte from an animal of the same species from which the quiescent cell is isolated. The reconstructed oocyte then is cultured such that it develops to morula or blastocyst and then transferred to a pseudopregnant female foster animal. Alternatively, a nucleus can be transferred to an enucleated host cell. The offspring borne of that female foster animal will be a clone of the animal from which the cell, e.g., the somatic cell, is isolated.

### IV. Pharmaceutical Compositions

The ZAC nucleic acid molecules, ZAC proteins, ZAC modulators and anti-ZAC antibodies (also referred to herein as “active compounds”) of the invention can be incorporated into pharmaceutical compositions suitable for administration. Such compositions typically comprise the nucleic acid molecule, protein, modulator or antibody and a pharmaceutically acceptable carrier. A “modulator” is a molecule or entity that causes a change in the structure or function of ZAC, such as zinc. As used herein, the language, “pharmaceutically acceptable carrier,” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds also can be incorporated into the compositions.

A pharmaceutical composition of the invention is formulated to be compatible with the intended route of administration. Examples of routes of administration include parenteral, e.g., intravenous, intradermal, subcutaneous, oral (e.g., inhalation), transdermal (topical), transmucosal and rectal administration. Solutions or suspensions used for parenteral, intradermal or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose. Acidity (pH) can be adjusted with acids or bases, such as HCl or NaOH. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL® (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyetheylene glycol and the like) and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a ZAC protein, ZAC modulator or anti-ZAC antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches or capsules. Oral compositions also can be prepared using a fluid carrier for use as a mouthwash, wherein the compound in the fluid carrier is applied orally, swished and expectorated or swallowed.

Pharmaceutically compatible binding agents and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel or corn starch; a lubricant such as magnesium stearate or Sterotes; a glidant such as colloidal silicon dioxide; a sweetening agent such as sucrose or saccharin; or a flavoring agent such as peppermint, methyl salicylate or orange flavoring. For administration by inhalation, the compounds are delivered in the form of an aerosol spray from a pressurized container or dispenser that contains a suitable propellant, e.g., a gas such as carbon dioxide, or a nebulizer.

Systemic administration also can be by transmucosal or transdermal means. For transmucosal or transdermal administration, penetrants appropriate to the barrier to be permeated are used in the formulation. Such penetrants generally are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels or creams, as generally known in the art.

The compounds also can be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid. Methods for preparing such formulations will be apparent to those skilled in the art. The materials also can be obtained commercially from Alza Corporation and Nova Pharmaceuticals, Inc. Liposomal suspensions (including liposomes targeted to infected cells with monoclonal antibodies to viral antigens) also can be used as pharmaceutically acceptable carriers. Those can be prepared according to methods known to those skilled in the art, for example, as described in U.S. Pat. No. 4,522,811.

It is especially advantageous to formulate oral or parenteral compositions in dosage unit form for ease of administration and uniformity of dosage. “Dosage unit form” as used herein refers to physically discrete units suited to unitary dosages, each unit containing a predetermined quantity of active compound calculated to produce the desired therapeutic effect in association with the required pharmaceutical carrier. Depending on the type and severity of the disease, about 1 μg/kg to 15 mg/kg (e.g., 0.1 to 20 mg/kg) of antibody is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of the therapy is monitored easily by conventional techniques and assays. An exemplary dosing regimen is disclosed in WO 94/04188. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see, e.g., Chen et al., Proc Natl Acad Sci USA (1994) 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack or dispenser, together with instructions for administration.

### V. Uses and Methods of the Invention

The nucleic acid molecules, proteins, protein homologues, modulators and antibodies described herein can be used in one or more of the following methods: a) screening assays; b) detection assays (e.g., chromosomal mapping, tissue typing, forensic biology); c) predictive medicine (e.g., diagnostic assays, prognostic assays, monitoring clinical trials and pharmacogenomics); and d) methods of treatment (e.g., therapeutic and prophylactic). A ZAC protein interacts with other cellular proteins via ion conductance and can thus be used for (i) regulation of cellular activation; (ii) regulation of cellular differentiation; and (iii) regulation of cell survival. The isolated nucleic acid molecules of the invention can be used to express ZAC protein (e.g., via a recombinant expression vector in a host cell in gene therapy applications), to detect ZAC mRNA (e.g., in a biological sample) or a genetic lesion in a ZAC gene, and to modulate ZAC activity. In addition, the ZAC proteins can be used to screen drugs or compounds which modulate the ZAC activity or expression as well as to treat disorders characterized by insufficient or excessive production of ZAC protein or production of ZAC protein forms which have decreased or aberrant activity compared to ZAC wild-type protein. In addition, the anti-ZAC antibodies of the invention can be used to detect and to isolate ZAC proteins and to modulate ZAC activity. The invention further pertains to novel agents identified by the above-described screening assays and uses thereof for treatments as described herein.

A. Screening Assays

The invention provides a method (also referred to herein as a “screening assay”) for identifying modulators, i.e., candidate or test compounds or agents (e.g., peptides, peptidomimetics, small molecules or other drug candidates) which bind to ZAC or have a stimulatory or inhibitory effect on, for example, ZAC expression or ZAC activity.

In one embodiment, the invention provides assays for screening candidate or test compounds that bind to or modulate the activity of the membrane-bound form of a ZAC protein or polypeptide or biologically active portion thereof. The test compounds of the instant invention can be obtained using any of the numerous approaches in combinatorial library methods known in the art, including: biological libraries; collections of synthesized compounds having related structures or not; spatially addressable parallel solid phase or solution phase libraries; synthetic library methods requiring deconvolution; the “one-bead one-compound” library method; and synthetic library methods using affinity chromatography selection. The biological library approach can be limited to peptide libraries, while the other four approaches can be applicable to peptide, non-peptide oligomer or small molecule libraries of compounds (Lam, Anticancer Drug Des (1997) 12:145).

Examples of methods for the synthesis of molecular libraries can be found in the art, for example in: DeWitt et al., Proc Natl Acad Sci USA (1993) 90:6909; Erb et al., Proc Natl Acad Sci USA (1994) 91:11422; Zuckermann et al., J Med Chem (1994) 37:2678; Cho et al., Science (1993) 261:1303; Carrell et al., Angew Chem Int Ed Engl (1994) 33:2059; Carell et al., Angew Chem Int Ed Engl (1994) 33:2061; and Gallop et al., J Med Chem (1994) 37:1233.

Libraries of compounds may be presented in solution (e.g., Houghten, Bio/Techniques (1992) 13:412-421), or on beads (Lam, Nature (1991) 354:82-84), chips (Fodor, Nature (1993) 364:555-556), bacteria (U.S. Pat. No. 5,223,409), spores (U.S. Pat. Nos. 5,571,698; 5,403,484; and 5,223,409), plasmids (Cull et al., Proc Natl Acad Sci USA (1992) 89:1865-1869) or phage (Scott et al., Science (1990) 249:386-390; Devlin, Science (1990) 249:404-406; Cwirla et al., Proc Natl Acad Sci USA (1990) 87:6378-6382; and Felici, J Mol Biol (1991) 222:301-310).

Because a ZAC modulator is zinc, zinc can be investigated to determine what particular portion of ZAC engages zinc, practicing known methods. That particular region can be synthesized practicing known biosynthetic methods, combining carbohydrate synthesis and enzymatic reactions, for example. That structure then can be used to determine the fine structure of the relevant site and that information can be used to predict structures that can engage and mimic the effects of zinc. Those discovered structures are test compounds or drug candidates.

In one embodiment, an assay is a cell-based assay in which a cell which expresses a membrane-bound form of ZAC protein, or a biologically active portion thereof, on the cell surface is contacted with a test compound and the ability of the test compound to bind to a ZAC protein determined. The cell, for example, can be a yeast cell or a cell of mammalian origin. Determining the ability of the test compound to bind to the ZAC protein can be accomplished, for example, by coupling the test compound with a radioisotope or enzymatic label such that binding of the test compound to the ZAC protein or biologically active portion thereof can be determined by detecting the labeled compound in a complex. For example, test compounds can be labeled with ¹²⁵I, ³⁵S, ¹⁴C or ³H, either directly or indirectly, and the radioisotope detected by direct counting of radioemmission or by scintillation counting. Alternatively, test compounds can be labeled enzymatically with, for example, horseradish peroxidase, alkaline phosphatase or luciferase, and the enzymatic label detected by determination of conversion of an appropriate substrate to product. In a preferred embodiment, the assay comprises contacting a cell which expresses a membrane-bound form of ZAC protein, or a biologically active portion thereof, on the cell surface with a known compound which binds ZAC to form an assay mixture, contacting the assay mixture with a test compound, and determining the ability of the test compound to interact with a ZAC protein, wherein determining the ability of the test compound to interact with a ZAC protein comprises determining the ability of the test compound to preferentially bind to ZAC or a biologically active portion thereof as compared to the known compound.

In another embodiment, an assay is a cell-based assay comprising contacting a cell expressing a membrane-bound form of ZAC protein, or a biologically active portion thereof, on the cell surface with a test compound and determining the ability of the test compound to modulate (e.g., stimulate or inhibit) the activity of the ZAC protein or biologically active portion thereof. Determining the ability of the test compound to modulate the activity of ZAC or a biologically active portion thereof can be accomplished, for example, by determining the ability of the ZAC protein to bind to or to interact with a ZAC target molecule. As used herein, a “target molecule” is a molecule with which a ZAC protein binds or interacts in nature, for example, a molecule on the surface of a cell which expresses a ZAC protein, a molecule on the surface of a second cell, a molecule in the extracellular milieu, a molecule associated with the internal surface of a cell membrane or a cytoplasmic molecule. A ZAC target molecule can be a non-ZAC molecule or a ZAC protein or polypeptide of the instant invention. In one embodiment, a ZAC target molecule is a component of a signal transduction pathway that facilitates transduction of an extracellular signal (e.g., a signal generated by binding of a compound to a membrane-bound ZAC molecule) through the cell membrane and into the cell. The target, for example, can be a second intercellular protein that has catalytic activity or a protein that facilitates the association of downstream signaling molecules with ZAC.

In another embodiment, ZAC is made to signal constitutively using known techniques, see, for example WO 00/22131 and WO 00/22129, expressed in a target cell as taught herein, and then the cell is exposed to various candidate modulators to determine if signaling activity, the monitoring of which is described herein, is enhanced, revealing a candidate agonist, or diminished, revealing a candidate antagonist, or if activity is reduced below baseline levels, a candidate inverse agonist.

Determining the ability of the ZAC protein to bind to or to interact with a ZAC target molecule can be accomplished by one of the methods described above for determining direct binding. In a preferred embodiment, determining the ability of the ZAC protein to bind to or to interact with a ZAC target molecule can be accomplished by determining the activity of the target molecule. For example, the activity of the target molecule can be determined by detecting induction of a current in the cell due to a transmembrane flux of ions.

In yet another embodiment, an assay of the instant invention is a cell-free assay comprising contacting a ZAC protein or biologically active portion thereof with a test compound and determining the ability of the test compound to bind to the ZAC protein or biologically active portion thereof. Binding of the test compound to the ZAC protein can be determined either directly or indirectly as described above. In a preferred embodiment, the assay includes contacting the ZAC protein or biologically active portion thereof with a known compound which binds ZAC to form an assay mixture, contacting the assay mixture with a test compound, and determining the ability of the test compound to interact with a ZAC protein, wherein determining the ability of the test compound to interact with a ZAC protein comprises determining the ability of the test compound to preferentially bind to ZAC or a biologically active portion thereof, as compared to the known compound.

In another embodiment, an assay is a cell-free assay comprising contacting ZAC protein or biologically active portion thereof with a test compound and determining the ability of the test compound to modulate (e.g., stimulate or inhibit) the activity of the ZAC protein or a biologically active portion thereof. Determining the ability of the test compound to modulate the activity of ZAC can be accomplished, for example, by determining the ability of the ZAC protein to bind to a ZAC target molecule by one of the methods described above for determining direct binding. In an alternative embodiment, determining the ability of the test compound to modulate the activity of ZAC can be accomplished by determining the ability of the ZAC protein to further modulate a ZAC target molecule. For example, the catalytic/enzymatic activity of the target molecule on an appropriate substrate can be determined as described previously.

In yet another embodiment, the cell-free assay comprises contacting the ZAC protein or biologically active portion thereof with a known compound which binds ZAC to form an assay mixture, contacting the assay mixture with a test compound, and determining the ability of the test compound to interact with a ZAC protein, wherein determining the ability of the test compound to interact with a ZAC protein comprises determining the ability of the ZAC protein to preferentially bind to or modulate the activity of a ZAC target molecule.

The cell-free assays of the instant invention are amenable to use of both the soluble form and the membrane-bound form of ZAC. In the case of cell-free assays comprising the membrane-bound form of ZAC, it may be desirable to utilize a solubilizing agent such that the membrane-bound form of ZAC is maintained in solution. Examples of such solubilizing agents include non-ionic detergents such as n-octylglucoside, Thesit®, n-dodecylglucoside, n-dodecylmaltoside, octanoyl-N-methylglucamide, decanoyl-N-methylglucamide, isotridecylpoly(ethyleneglycol-ether)ₙ, Triton X-100, Triton X-114, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO) or N-dodecyl═N,N-dimethyl-3-ammonio-1-propane sulfonate.

In more than one embodiment of the above assay methods of the instant invention, it may be desirable to immobilize either ZAC or a target molecule to facilitate separation of complexed from uncomplexed forms of one or both of the reagents, as well as to accommodate automation of the assay. Binding of a test compound to ZAC, or interaction of ZAC with a target molecule in the presence and absence of a candidate compound, can be accomplished in any vessel suitable for containing the reactants. Examples of such vessels include microtitre plates, test tubes and micro-centrifuge tubes. In one embodiment, a fusion protein can be provided which adds a domain that allows one or both of the proteins to be bound to a matrix. For example, glutathione-S-transferase/ZAC fusion proteins or glutathione-S-transferase/target fusion proteins can be adsorbed onto glutathione Sepharose beads (Sigma Chemical, St. Louis, Mo.) or glutathione derivatized microtitre plates, which then are combined with the test compound or the test compound and either the non-adsorbed target protein or ZAC protein, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix and the level of ZAC binding or activity determined using standard techniques.

Other techniques for immobilizing proteins on matrices also can be used in the screening assays of the invention. For example, either ZAC or its target molecule can be immobilized utilizing conjugation of biotin and streptavidin. Biotinylated ZAC or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemicals). Alternatively, antibodies reactive with ZAC or target molecules but which do not interfere with binding of the ZAC protein to a target molecule can be derivatized to the wells of the plate, and unbound target or ZAC trapped in the wells by antibody conjugation. Methods for detecting such complexes, in addition to those described above for the GST-immobilized complexes, include immunodetection of complexes using antibodies reactive with the ZAC or target molecule, as well as enzyme-linked assays which rely on detecting an enzymatic activity associated with the ZAC or target molecule.

In another embodiment, modulators of ZAC expression are identified in a method in which a cell is contacted with a candidate compound and the expression of ZAC mRNA or protein in the cell is determined. The level of expression of ZAC mRNA or protein in the presence of the candidate compound is compared to the level of expression of ZAC mRNA or protein in the absence of the candidate compound. The candidate compound then can be identified as a modulator of ZAC expression based on that comparison. For example, when expression of ZAC mRNA or protein is greater (statistically significantly greater) in the presence of the candidate compound than in its absence, the candidate compound is identified as a stimulator of ZAC mRNA or protein expression. Alternatively, when expression of ZAC mRNA or protein is less (statistically significantly less) in the presence of the candidate compound than in its absence, the candidate compound is identified as an inhibitor of ZAC mRNA or protein expression. The level of ZAC mRNA or protein expression in the cells can be determined by methods described herein for detecting ZAC mRNA or protein.

In yet another aspect of the invention, the ZAC proteins can be used as “bait proteins” in a two-hybrid assay or three-hybrid assay (see, e.g., U.S. Pat. No. 5,283,317; Zervos et al., Cell (1993) 72:223-232; Madura et al., J Biol Chem (1993) 268:12046-12054; Bartel et al., Bio/Techniques (1993) 14:920-924; Iwabuchi et al., OncoGene (1993) 8:1693-1696; and PCT Publication No. WO 94/10300), to identify other proteins, which bind to or interact with ZAC (“ZAC-binding proteins” or “ZAC-bp”) and modulate ZAC activity. Such ZAC-binding proteins are also likely to be involved in the propagation of signals by the ZAC proteins as, for example, upstream or downstream elements of the ZAC pathway.

The invention further pertains to novel agents identified by the above-described screening assays and uses thereof for treatments as described herein.

B. Detection Assays

Portions or fragments of the cDNA sequences identified herein (and the corresponding complete gene sequences) can be used in numerous ways as polynucleotide reagents. For example, the sequences can be used to: (i) map respective genes on a chromosome and, thus, locate gene regions associated with genetic disease; (ii) identify an individual from a minute biological sample (tissue typing); and (iii) aid in forensic identification of a biological sample. Those applications are described in the subsections below.

1. Chromosome Mapping

ZAC nucleic acid molecules described herein or fragments thereof; can be used to therefore investigate the sequences about ZAC genes on chromosome 17 and more specifically 17q23. The mapping of the ZAC sequences to chromosome 17 is an important step in correlating the sequences with genes associated with disease.

The relationship between genes and disease, mapped to the same chromosomal region, then can be identified through linkage analysis (co-inheritance of physically adjacent genes), described in, e.g., Egeland et al., Nature (1987) 325:783-787.

Moreover, differences in the DNA sequences between individuals affected and unaffected with a disease associated with the ZAC gene can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The ZAC sequences of the instant invention also can be used to identify individuals from minute biological samples. The United States military, for example, is considering the use of restriction fragment length polymorphism (RFLP) for identification of personnel. In that technique, genomic DNA of an individual is digested with one or more restriction enzymes, and probed on a Southern blot to yield unique bands for identification. The method does not suffer from the current limitations of “Dog Tags” which can be lost, switched or stolen, making positive identification difficult. The sequences of the instant invention are useful as additional DNA markers for RFLP (described in U.S. Pat. No. 5,272,057).

3. Use of Partial ZAC Sequences in Forensic Biology

DNA-based identification techniques also can be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva or semen, found at a crime scene. The amplified sequence then can be compared to a standard, thereby allowing identification of the origin of the biological sample.

The sequences of the instant invention can be used to provide polynucleotide reagents, e.g., PCR primers, targeted to specific loci in the human genome, which can enhance the reliability of DNA-based forensic identifications by, for example, providing another “identification marker” (i.e., another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1 are particularly appropriate for that use as greater numbers of polymorphisms occur in the noncoding regions, making it easier to differentiate individuals using that technique. Examples of polynucleotide reagents include the ZAC sequences or portions thereof, e.g., fragments derived from the noncoding regions of SEQ ID NO:1 having a length of at least 20 or 30 bases.

In a similar fashion, the reagents, e.g., ZAC primers or probes can be used to screen tissue culture for contamination (i.e., screen for the presence of a mixture of different types of cells in a culture).

4. Biosensors

Cells expressing ZAC, membranes containing ZAC, supports, naturally occurring or artificial, carrying ZAC or ZAC per se can be used as an absorbent or detector of zinc and other cations. A sample suspected of containing zinc or other molecule that binds or interacts with ZAC is contacted with a ZAC and binding or interaction determined, as taught herein.

C. Predictive Medicine

The instant invention also pertains to the field of predictive medicine in which diagnostic assays, prognostic assays, pharmacogenomics and monitoring clinical trails are used for prognostic (predictive) purposes to treat an individual prophylactically. Accordingly, one aspect of the instant invention relates to diagnostic assays for determining ZAC protein and/or nucleic acid expression as well as ZAC activity, in the context of a biological sample (e.g., blood, urine, feces, serum, cells, tissue) to determine whether an individual is afflicted with a disease or disorder, or is at risk of developing a disorder, associated with aberrant ZAC expression or activity. The invention also provides for prognostic (or predictive) assays for determining whether an individual is at risk of developing a disorder associated with ZAC protein, nucleic acid expression or activity. For example, mutations in a ZAC gene can be assayed in a biological sample. Such assays can be used for prognostic or predictive purpose to thereby prophylactically treat an individual prior to the onset of a disorder characterized by or associated with ZAC protein, nucleic acid expression or activity.

Another aspect of the invention provides methods for determining ZAC protein, nucleic acid expression or ZAC activity in an individual to select thereby appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent).

Yet another aspect of the invention pertains to monitoring the influence of agents (e.g., drugs or other compounds) on the expression or activity of ZAC in clinical trials.

Those and other agents are described in further detail in the following sections.

1. Diagnostic Assays

An exemplary method for detecting the presence or absence of ZAC in a biological sample involves obtaining a biological sample from a test subject and contacting the biological sample with a compound or an agent capable of detecting ZAC protein or nucleic acid (e.g., mRNA, genomic DNA molecule that binds ZAC or modulates ZAC activity) that encodes ZAC protein such that the presence of ZAC is detected in the biological sample. A preferred agent for detecting ZAC mRNA or genomic DNA is a labeled nucleic acid probe capable of hybridizing to ZAC mRNA or genomic DNA. The nucleic acid probe can be, for example, a full-length ZAC nucleic acid, such as the nucleic acid of SEQ ID NO:1 or a portion thereof, such as an oligonucleotide of at least 15, 30, 50, 100, 250 or 500 nucleotides in length and sufficient to specifically hybridize under stringent conditions to ZAC mRNA or genomic DNA. Other suitable probes for use in the diagnostic assays of the invention are described herein.

A suitable agent for detecting ZAC protein is an antibody capable of binding to ZAC protein, preferably an antibody with a detectable label. Antibodies can be polyclonal, or more preferably, monoclonal. An intact antibody, or a fragment thereof (e.g., F_{ab} or F_{(ab′)2}) can be used. The term, “labeled”, with regard to the antibody, is intended to encompass direct labeling of the antibody by coupling (i.e., physically linking) a detectable substance to the antibody, as well as indirect labeling of the antibody by reactivity with another reagent that is directly labeled. Examples of indirect labeling include detection of a primary antibody using a fluorescently labeled secondary antibody or labeling of an antibody with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect ZAC mRNA, protein or genomic DNA in a biological sample. Sitable techniques for detecting ZAC mRNA include Northern hybridization, in situ hybridization, enzyme linked immunosorbent assay (ELISAs), Western blot, immunoprecipitation and immunofluorescence. In vitro techniques for detection of ZAC genomic DNA include Southern hybridizations. Furthermore, in vivo techniques for detection of ZAC protein include introducing into a subject a labeled anti-ZAC antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting ZAC protein, mRNA or genomic DNA, such that the presence of ZAC protein, mRNA or genomic DNA is detected in the biological sample, and comparing the presence of ZAC protein, mRNA or genomic DNA in the control sample with the presence of ZAC protein, mRNA or genomic DNA in the test sample.

The invention also encompasses kits for detecting the presence of ZAC in a biological sample (a test sample). Such kits can be used to determine if a subject is suffering from or is at increased risk of developing a disorder associated with aberrant expression of ZAC. For example, the kit can comprise a labeled compound or agent capable of detecting ZAC protein or mRNA in a biological sample and means for determining the amount of ZAC in the sample (e.g., an anti-ZAC antibody or an oligonucleotide probe which binds to DNA encoding ZAC, e.g., SEQ ID NO:1). Kits also can include instructions for observing that the tested subject is suffering from or is at risk of developing a disorder associated with aberrant expression of ZAC, if the amount of ZAC protein or mRNA is above or below a normal level.

For antibody-based kits, the kit can comprise, for example: (1) a first antibody (e.g., attached to a solid support) which binds to ZAC protein; and, optionally, (2) a second, different antibody which binds to ZAC protein or the first antibody and is conjugated to a detectable agent.

For oligonucleotide-based kits, the kit can comprise, for example: (1) an oligonucleotide, e.g., a detectably labeled oligonucleotide, which hybridizes to a ZAC nucleic acid sequence or (2) a pair of primers useful for amplifying a ZAC nucleic acid molecule.

The kit also can comprise, e.g., a buffering agent, a preservative or a protein stabilizing agent. The kit also can comprise components necessary for detecting the detectable agent (e.g., an enzyme or a substrate). The kit also can contain a control sample or a series of control samples that can be assayed and compared to the test sample contained. Each component of the kit usually is enclosed within an individual container and all of the various containers are within a single package along with instructions for observing whether the tested subject is suffering from or is at risk of developing a disorder associated with aberrant expression of ZAC.

2. Prognostic Assays

The methods described herein furthermore can be utilized as diagnostic or prognostic assays to identify subjects having or at risk of developing a disease or disorder associated with aberrant ZAC expression or activity. For example, the assays described herein, such as the preceding diagnostic assays or the following assays, can be utilized to identify a subject having or at risk of developing a disorder associated with ZAC protein, nucleic acid expression or activity. Alternatively, the prognostic assays can be utilized to identify a subject having or at risk of developing such a disease or disorder. Thus, the instant invention provides a method in which a test sample is obtained from a subject and ZAC protein or nucleic acid (e.g., mRNA, genomic DNA) is detected, wherein the presence of ZAC protein or nucleic acid is diagnostic for a subject having or at risk of developing a disease or disorder associated with aberrant ZAC expression or activity. As used herein, a “test sample” refers to a biological sample obtained from a subject of interest. For example, a test sample can be a biological fluid (e.g., serum), cell sample or tissue. Furthermore, the prognostic assays described herein can be used to determine whether a subject can be administered an agent (e.g., an agonist, antagonist, peptidomimetic, protein, peptide, nucleic acid, small molecule or other drug candidate) to treat a disease or disorder associated with aberrant ZAC expression or activity. For example, such methods can be used to determine whether a subject can be treated effectively with a specific agent or class of agents (e.g., agents of a type that decrease ZAC activity). Thus, the instant invention provides methods for determining whether a subject can be treated effectively with an agent for a disorder associated with aberrant ZAC expression or activity in which a test sample is obtained and ZAC protein or nucleic acid is detected (e.g., wherein the presence of ZAC protein or nucleic acid is diagnostic for a subject that can be administered the agent to treat a disorder associated with aberrant ZAC expression or activity).

The methods of the invention also can be used to detect genetic lesions or mutations in a ZAC gene, thereby determining if a subject with the lesioned gene is at risk for a disorder characterized by aberrant cell proliferation and/or differentiation. In preferred embodiments, the methods include detecting, in a sample of cells from the subject, the presence or absence of a genetic lesion or mutation characterized by at least one alteration affecting the integrity of a gene encoding a ZAC protein, or the misexpression of the ZAC gene. For example, such genetic lesions or mutations can be detected by ascertaining the existence of at least one of: 1) a deletion of one or more nucleotides from a ZAC gene; 2) an addition of one or more nucleotides to a ZAC gene; 3) a substitution of one or more nucleotides of a ZAC gene; 4) a chromosomal rearrangement of a ZAC gene; 5) an alteration in the level of a messenger RNA transcript of a ZAC gene; 6) an aberrant modification of a ZAC gene, such as of the methylation pattern of the genomic DNA; 7) the presence of a non-wild-type splicing pattern of a messenger RNA transcript of a ZAC gene; 8) a non-wild-type level of a ZAC protein; 9) an allelic loss of a ZAC gene; and 10) an inappropriate post-translational modification of a ZAC protein. As described herein, there are a large number of assay techniques known in the art that can be used for detecting lesions in a ZAC gene. A preferred biological sample is a peripheral blood leukocyte sample isolated by conventional means from a subject.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al., Science (1988) 241:1077-1080; and Nakazawa et al., Proc Natl Acad Sci USA (1994) 91:360-364), the latter of which can be particularly useful for detecting point mutations in the ZAC gene (see, e.g., Abravaya et al., Nucleic Acids Res (1995) 23:675-682). The method can include the steps of collecting a sample of cells from a patient, isolating nucleic acid (e.g., genomic, mRNA or both) from the cells of the sample, contacting the nucleic acid sample with one or more primers which specifically hybridize to a ZAC gene under conditions such that hybridization and amplification of the ZAC gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al., Proc Natl Acad Sci USA (1990) 87:1874-1878), transcriptional amplification system (Kwoh et al., Proc Natl Acad Sci USA (1989) 86:1173-1177), Q-β Replicase (Lizardi et al., Bio/Technology (1988) 6:1197), or any other nucleic acid amplification method, followed by the detection of the amplified molecules using techniques well known to those of skill in the art. The detection schemes are especially useful for the detection of nucleic acid molecules if such molecules are present in very low numbers.

In an alternative embodiment, mutations in a ZAC gene from a sample cell can be identified by alterations in restriction enzyme cleavage patterns. For example, sample and control DNA are isolated, amplified (optionally), digested with one or more restriction endonucleases, and fragment length sizes are determined by gel electrophoresis and compared. Differences in fragment length sizes between sample and control DNA indicate mutations in the sample DNA. Moreover, the use of sequence-specific ribozymes (see, e.g., U.S. Pat. No. 5,498,531) can be used to score for the presence of specific mutations by development or loss of a ribozyme cleavage site.

In other embodiments, genetic mutations in ZAC can be identified by hybridizing a sample and control nucleic acids, e.g., DNA or RNA, to high density arrays containing hundreds or thousands of oligonucleotides probes (Cronin et al., Human Mutation (1996) 7:244-255; KoZAC et al., Nature Medicine (1996) 2:753-759). For example, genetic mutations in ZAC can be identified in two-dimensional arrays containing light-generated DNA probes as described in Cronin et al., supra. Briefly, a first hybridization array of probes can be used to scan through long stretches of DNA in a sample and to identify base changes between the sequences by making linear arrays of sequential overlapping probes. That step allows the identification of point mutations. That step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the ZAC gene and to detect mutations by comparing the sequence of the sample ZAC with the corresponding wild-type (control) sequence. Examples of sequencing reactions include those based on techniques developed by Maxam & Gilbert (Proc Natl Acad Sci USA (1977) 74:560) or Sanger (Proc Natl Acad Sci USA (1977) 74:5463). It also is contemplated that any of a variety of automated sequencing procedures can be utilized when performing the diagnostic assays (Bio/Techniques (1995) 19:448), including sequencing by mass spectrometry (see, e.g., PCT Publication No. WO 94/16101; Cohen et al., Adv Chromatogr (1996) 36:127-162; and Griffin et al., Appl Biochem Biotechnol (1993) 38:147-159).

Other methods for detecting mutations in the ZAC gene include methods in which protection from cleavage agents is used to detect mismatched bases in RNA/RNA or RNA/DNA heteroduplexes (Myers et al., Science (1985) 230:1242). In general, the technique of “mismatch cleavage” entails providing heteroduplexes formed by hybridizing (labeled) RNA or DNA containing the wild-type ZAC sequence with potentially mutant RNA or DNA obtained from a tissue sample. The double-stranded duplexes are treated with an agent that cleaves single-stranded regions of the duplex such will exist due to base pair mismatches between the control and sample strands. RNA/DNA duplexes can be treated with RNase to digest mismatched regions, and DNA/DNA hybrids can be treated with S1 nuclease to digest mismatched regions. In other embodiments, either DNA/DNA or RNA/DNA duplexes can be treated with hydroxylamine or osmium tetroxide and with piperidine to digest mismatched regions. After digestion of the mismatched regions, the resulting material then is separated by size on denaturing polyacrylamide gels to determine the site of mutation, see, e.g., Cotton et al., Proc Natl Acad Sci USA (1988) 85:4397; Saleeba et al., Methods Enzymol (1992) 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in ZAC cDNAs obtained from samples of cells. For example, the mutY enzyme of E. coli cleaves A at G/A mismatches and the thymidine DNA glycosylase from HeLa cells cleaves T at G/T mismatches (Hsu et al., Carcinogenesis (1994) 15:1657-1662). According to an exemplary embodiment, a probe based on a ZAC sequence, e.g., a wild-type ZAC sequence, is hybridized to a cDNA or other DNA product from a test cell(s). The duplex is treated with a DNA mismatch repair enzyme, and the cleavage products, if any, can be detected from electrophoresis protocols or the like, see, e.g., U.S. Pat. No. 5,459,039.

In other embodiments, alterations in electrophoretic mobility will be used to identify mutations in ZAC genes. For example, single strand conformation polymorphism (SSCP) may be used to detect differences in electrophoretic mobility between mutant and wild-type nucleic acids (Orita et al., Proc Natl Acad Sci USA (1989) 86:2766; see also Cotton, Mutat Res (1993) 285:125-144; Hayashi, Genet Anal Tech Appl (1992) 9:73-79). Single-stranded DNA fragments of sample and control ZAC nucleic acids will be denatured and allowed to renature. The secondary structure of single-stranded nucleic acids varies according to sequence, and the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double-stranded heteroduplex molecules on the basis of changes in electrophoretic mobility (Keen et al., Trends Genet (1991) 7:5).

In yet another embodiment, the movement of mutant or wild-type fragments in polyacrylamide gels containing a gradient of denaturant is assayed using denaturing gradient gel electrophoresis (DGGE) (Myers et al., Nature (1985) 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum et al., Biophys Chem (1987) 265:12753).

Examples of other techniques for detecting point mutations include, but are not limited to, selective oligonucleotide hybridization, selective amplification or selective primer extension. For example, oligonucleotide primers may be prepared in which the known mutation is placed centrally and then hybridized to target DNA under conditions which permit hybridization only if a perfect match is found (Saiki et al., Nature (1986) 324:163); Saiki et al., Proc Natl Acad Sci USA (1989) 86:6230). Such allele-specific oligonucleotides are hybridized to PCR-amplified target DNA or a number of different mutations when the oligonucleotides are attached to the hybridizing membrane and hybridized with labeled target DNA.

Alternatively, allele-specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al., Nucleic Acids Res (1989) 17:2437-2448) or at the extreme 3′ end of one primer where, under appropriate conditions, mismatch can prevent or reduce polymerase extension (Prossner, Tibtech (1993) 11:238). In addition, it may be desirable to introduce a novel restriction site in the region of the mutation to create cleavage-based detection (Gasparini et al., Mol Cell Probes (1992) 6:1). It is anticipated that in certain embodiments amplification also may be performed using Taq ligase for amplification (Barany, Proc Natl Acad Sci USA (1991) 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be used conveniently, e.g., in clinical settings to diagnose patients exhibiting symptoms or family history of a disease or illness involving a ZAC gene.

Furthermore, any cell type or tissue where ZAC is expressed may be utilized in the prognostic assays described herein.

3. Pharmacogenomics

Agents, or modulators that have a stimulatory or inhibitory effect on ZAC activity (e.g., ZAC gene expression or ZAC activity) as identified by a screening assay described herein, can be administered to individuals to treat (prophylactically or therapeutically) disorders, such as neurotransmitter-modulated disorders in the stomach (e.g., gastrinoma, gastric ulcers), spinal cord (e.g., ataxia), trachea (e.g., croup, allergic edema) and thyroid (e.g., hypothyroidism, hyperthyroidism), associated with ZAC activity. In conjunction with such treatment, the pharmacogenomics (i.e., the study of the relationship between the genotype of an individual and the response of that individual to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the genotype of an individual. Such pharmacogenomics further can be used to determine appropriate dosages and therapeutic regimens. Accordingly, the activity of ZAC protein, expression of ZAC nucleic acid or mutation content of ZAC genes in an individual can be determined thereby to select appropriate agent(s) for therapeutic or prophylactic treatment of the individual.

Pharmacogenomics deals with clinically significant hereditary variations in the response to drugs due to altered drug disposition and abnormal action in affected persons, see, e.g., Linder, Clin Chem (1997) 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism.” The pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes, CYP2D6 and CYP2Cl9) has provided an explanation as to why some patients do not obtain the expected drug effects or show exaggerated drug response and serious toxicity after taking the standard and safe dose of a drug. The polymorphisms are expressed in two phenotypes in the population, the extensive metabolizer (EM) and the poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2Cl9 quite frequently experience exaggerated drug response and side effects when they receive standard doses. If a metabolite is the active therapeutic moiety, a PM will show no therapeutic response, as demonstrated for the analgesic effect of codeine mediated by the CYP2D6-formed metabolite, morphine. The other extreme is the so called ultra-rapid metabolizers that do not respond to standard doses. Recently, the molecular basis of ultra-rapid metabolism has been identified to be due to CYP2D6 gene amplification.

Thus, the activity of ZAC protein, expression of ZAC nucleic acid or mutation content of ZAC genes in an individual can be determined to thereby select appropriate agent(s) for therapeutic or prophylactic treatmnent of the individual. In addition, pharmacogenetic studies can be used to apply genotyping of polymorphic alleles encoding the drug-metabolizing enzymes to the identification of the drug responsiveness phenotype of an individual. That knowledge, when applied to dosing or drug selection, can avoid adverse reactions or therapeutic failure and thus enhance therapeutic or prophylactic efficiency when treating a subject with a ZAC modulator, such as a modulator identified by one of the exemplary screening assays described herein.

4. Monitoring of Effects During Clinical Trials

Monitoring the influence of agents (e.g., drugs, compounds) on the expression or activity of ZAC (e.g., the ability to modulate aberrant cell proliferation and/or differentiation) can be applied not only in basic drug screening, but also in clinical trials. For example, the effectiveness of an agent, as determined by a screening assay as described herein, to increase ZAC gene expression, protein levels or protein activity, can be monitored in clinical trials of subjects exhibiting decreased ZAC gene expression, protein levels or protein activity. Alternatively, the effectiveness of an agent, as determined by a screening assay, to decrease ZAC gene expression, protein levels or protein activity, can be monitored in clinical trials of subjects exhibiting increased ZAC gene expression, protein levels or protein activity. In such clinical trials, ZAC expression or activity and preferably, that of other genes that have been implicated in, for example, a cellular proliferation disorder, can be used as a marker of the immune responsiveness of a particular cell.

For example, and not by way of limitation, genes, including ZAC, that are modulated in cells by treatment with an agent (e.g., compound, drug or small molecule) which modulates ZAC activity (e.g., as identified in a screening assay described herein) can be identified. Thus, to study the effect of agents on cellular proliferation disorders, for example, in a clinical trial, cells can be isolated and RNA prepared and analyzed for the levels of expression of ZAC and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of ZAC or other genes. In that way, the. gene expression pattern can serve as a marker, indicative of the physiological response of the cells to the agent. Accordingly, the response state may be determined before, and at various points during, treatment of the individual with the agent.

In a preferred embodiment, the instant invention provides a method for monitoring the effectiveness of treatment of a subject with an agent (e.g., an agonist, antagonist, peptidomimetic, protein, peptide, nucleic acid, small molecule or other drug candidate identified by the screening assays described herein) comprising the steps of: (i) obtaining a pre-administration sample from a subject prior to administration of the agent; (ii) detecting the level of expression of a ZAC protein, mRNA or genomic DNA in the preadministration sample; (iii) obtaining one or more post-administration samples from the subject; (iv) detecting the level of expression or activity of the ZAC protein, mRNA or genomic DNA in the post-administration samples; (v) comparing the level of expression or activity of the ZAC protein, mRNA or genomic DNA in the pre-administration sample with the ZAC protein, mRNA or genomic DNA in the post-administration sample or samples; and (vi) altering the administration of the agent to the subject accordingly. For example, increased administration of the agent may be desirable to increase the expression or activity of ZAC to higher levels than detected, i.e., to increase the effectiveness of the agent. Alternatively, decreased administration of the agent may be desirable to decrease expression or activity of ZAC to lower levels than detected, i.e., to decrease the effectiveness of the agent.

D. Methods of Treatment

The instant invention provides for both prophylactic and therapeutic methods of treating a subject at risk of (or susceptible to) a disorder or having a disorder associated with aberrant ZAC expression or activity, particularly those mapped to 17q23, such as Meckel syndrome, type 1; gene map locus 17q22-q23; and malignant hyperthermia susceptibility 2; 17q11.2-q24.

1. Prophylactic Methods

In one aspect, the invention provides a method for preventing in a subject, a disease or condition associated with an aberrant ZAC expression or activity, by administering to the subject an agent that modulates ZAC expression or at least one ZAC activity. Subjects at risk for a disease that is caused or contributed to by aberrant ZAC expression or activity can be identified by, for example, any or a combination of diagnostic or prognostic assays as described herein. Administration of a prophylactic agent can occur prior to the manifestation of symptoms characteristic of ZAC aberrancy, such that a disease or disorder is prevented or, alternatively, delayed in progression. Depending on the type of ZAC aberrancy, for example, a ZAC agonist or ZAC antagonist agent can be used for treating the subject. The appropriate agent can be determined based on screening assays described herein.

2. Therapeutic Methods

Another aspect of the invention pertains to methods of modulating ZAC expression or activity for therapeutic purposes. The modulatory method of the invention involves contacting a cell with an agent that modulates one or more of the activities of ZAC protein activity associated with the cell. An agent that modulates ZAC protein activity can be an agent as described herein, such as a nucleic acid or a protein, a naturally-occurring cognate ligand of a ZAC protein, a peptide, a ZAC peptidomimetic or other small molecule. The agent can be a agonist, inverse agonist or antagonist. In one embodiment, the agent stimulates one or more of the biological activities of ZAC. Examples of such stimulatory agents include active ZAC protein and a nucleic acid molecule encoding ZAC that has been introduced into the cell. In another embodiment, the agent inhibits one or more of the biological activities of ZAC. Examples of such inhibitory agents include antisense ZAC nucleic acid molecules and anti-ZAC antibodies. The modulatory methods can be performed in vitro (e.g., by culturing the cell with the agent) or, alternatively, in vivo (e.g., by administering the agent to a subject). As such, the instant invention provides methods of treating an individual afflicted with a disease or disorder characterized by aberrant expression or activity of a ZAC protein or nucleic acid molecule. In one embodiment, the method involves administering an agent (e.g., an agent identified by a screening assay described herein) or combination of agents that modulates (e.g., upregulates or downregulates) ZAC expression or activity. In another embodiment, the method involves administering a ZAC protein or nucleic acid molecule as therapy to compensate for reduced or aberrant ZAC expression or activity.

Stimulation of ZAC activity is desirable in situations in which ZAC is abnormally downregulated and/or in which increased ZAC activity is likely to have a beneficial effect. Conversely, inhibition of ZAC activity is desirable in situations in which ZAC is abnormally upregulated and/or in which decreased ZAC activity is likely to have a beneficial effect. Suitable ZAC modulators, agonists or antagonists will find use as therapeutic agents.

The invention is further illustrated by the following examples which should not be construed as limiting. The contents of all references, patents and published patent applications cited throughout the instant application hereby are incorporated by reference.

### EXAMPLE 1

### Cloning ZAC

Oligonucleotide primers were designed from the genomic sequence of interest to amplify the 5′ and 3′ flanking sequences from fetal brain and spinal cord cDNA libraries using the Marathon system (Clontech). Amplification at 95° C. for 45 s, 60° C. for 60 s, and 72° C. for 2 min was performed for 35 cycles using the XL-PCR system (PerkinElmer Life Sciences). The amplified sequence was purified from agarose gels and sequenced directly. The open reading frame of the ZAC cDNA was amplified from a spinal cord cDNA library using primers containing nucleotides 1-21 (sense) and 1266-1289 (antisense) of the ZAC subunit cDNA sequence (GenBank™ accession number AF512521). The cloned product was sequenced to ensure that no mutations had been introduced. Sequence alignments were generated by the ClustalW program from the MacVector package of sequence analysis software (Oxford Molecular Group). A cladogram was constructed using the neighbor-joining method with pairwise distances measured by absolute differences and gaps ignored. The bootstrap consensus was generated using 1,000 replications.

### EXAMPLE 2

### Generation of CHO Cels Overexpressing hZAC

To provide significant quantities of hZAC for further experiments, the cDNA encoding hZAC was cloned into an expression vector and transfected into human embryonic kidney (HEK) cells.

To generate HEK expressing hZAC, HEK cells were grown in Dulbecco's modified Eagle's medium, supplemented with 10% calf serum, 100 IU/ml penicillin, and 100 μg/ml streptomycin. Following confluence, the cells were seeded into 35 mm diameter dishes and transfected with cDNAs encoding the human ZAC subunit (in pcDNA1.1/amp) and green fluorescent protein (GFP) (in pCDM8). Cells were transfected using calcium phosphate precipitation as known in the art. Cells were used 24-44 h after transfection.

HEK cells transfected with and expressing ZAC displayed spontaneous currents immediately after achieving the whole cell configuration. Such recordings were not observed in cells transfected with GFP. Thus, ZAC forms patent ion channels.

GABA, glycine, glutamate, ATP, 5-HT, acetylcholine, galonin, epinephrine, dopamine, histamine, neuropeptide Y, oxytocin, morphine, somatostain, angiotensin II, glutathione, ketamine, allopregnanolone and propofol, known receptor agonists, did not activate ZAC.

Known antagonists, strychnine, bicuculline methiodide, α-bungarotoxin, mecamylamine and ondansetron had no effect on ZAC.

Tubocurarine, a non-selective inhibitor of nACH and 5-HT₃ receptors, inhibited ZAC.

Zn⁺² normally is an inhibitor of gated ion channels but activated ZAC. Zn⁺² activated currents had an equilibrium potential of −5±1 mV. Experiments revealed that intracellular K⁺ ions impacted current. The channels have negligible Cl⁻ permeability. Moreover, it appeared that Zn⁺² activates previously closed channels. A concentration of at least >30 μM Zn⁺² is requied for activation of ZAC.

Physiologically, Zn⁺² is concentrated in, for example, forebrain, testis and neuroendocrine cells. In the hippocampus, pituitary and pancreatic B cells, Zn⁺² is observed in vesicles at high concentration.

### EXAMPLE 3

### Electrophysiology

The whole cell patch-clamp technique was used to record currents from HEK cells. The bath was perfused continuously (5 ml/min) with an extracellular solution containing (in mM); NaCl, 140; KCl, 4.7; MgCl₂, 1.2; CaCl₂, 2.5; glucose, 11; and HEPES, 10 (pH 7.4 with NaOH). The electrode solution contained (in mM); KCl, 140; MgCl₂, 2.0; EGTA, 11; and HEPES, 10 (pH 7.4 with KOH). The intracellular solution used to characterize the cation permeability of ZAC channels contained (in mM); KCl, 70; N-methyl-D-glucamine, 70; MgCl₂, 2.0; EGTA, 11; and HEPES, 10 (pH 7.4 with HCl). The intracellular solution used to determine the contribution of Cl⁻ to the ZAC currents contained (in mM); KCl, 70; K⁺ gluconate, 70; MgCl₂, 2.0; EGTA, 11; and HEPES, 10 (pH 7.4 with KOH). Junction potentials were nulled prior to each experiment. Inappropriate inappropriate compensation was ignored in graphs of current-voltage relationships, but equilibrium potential values were corrected. Cells were clamped at −60 mV unless otherwise stated. Drugs were applied either by pressure ejection from modified micropipettes or by bath perfusion as known in the art. Experiments were performed at 22-24° C.

Currents were amplied (Axopatch 200A, Axon Instruments), low pass-filtered at 1 kHz, and digitized (Digidata 1320, Axon Instruments, Foster City, Calif.) for acquisition onto the hard drive of a personal computer. Currents were averaged, superimposed, and measured using pCLAMP software (Axon Instruments). Zn⁺² concentration-response data were obtained by prolonged (2 s) pressue ejection of randomized agonist concentrations from low resistance pipettes as known in the art.

Zn⁺² activated currents often exhibited run-up. To compensate, 1 mM Zn⁺² was applied before each concentration of Zn⁺². The amplitudes of the Zn⁺² activated currents were subsequently normalized to the current elicited by the prior application of 1 mM Zn⁺².

Graphs of concentration-response relationships were fitted using a logistic function as known in the art. Current-voltage relationships were analyzed by averaging at least two currents recorded at each holding potential. Individual current-voltage relationships were plotted, and a linear fit to points either side of current reversal yielded the equilibrium potential. All data are expressed as the arithmetic mean±S.E., and statistical comparisons were made using the Student's t test.

### EXAMPLE 4

### Northern Blot Analysis

Northern blot analysis was performed on RNA derived from several human tissue samples to determine whether the tissues express the hZAC receptor gene.

Samples of ˜2 μg of poly(A)⁺mRNA (Clontech) underwent electrophoresis on a 1.2% formaldehyde agarose gel, were transferred to nylon membranes, and were hybridized with an antisense ³²P-labeled riboprobe that was derived from the ZAC subunit cDNA (nucleotides 1-447 of GenBank™ accession number AF512521). The blots were washed at 60° C. in 0.1 ×SSC, 0.1% SDS before exposure. The blots were stripped and reprobed with ³²P-labeled fragments of the glyceraldehydes-3 phosphate dehydrogenase cDNA (nucleotides 789-1140) as a control.

hZAC is expressed in human placenta, trachea, spinal cord, stomach and fetal brain.

Although the instant invention has been described in detail with reference to the examples above, it is understood that various modifications can be made without departing from the spirit of the invention. Accordingly, the invention is limited only by the following claims.

All cited patents and publications referred to in this application are herein incorporated by reference in their entirety.

We claim:

1. An isolated nucleic acid comprising the nucleotide sequence of a zinc activated ion channel (ZAC) (SEQ ID NO:1) or a variant of ZAC activated by zinc.

2. The isolated nucleic acid of claim 1, wherein said sequence encodes a ZAC polypeptide with the amino acid sequence of SEQ ID NO:2.

3. The nucleic acid of claim 1, wherein said nucleic acid is selected from the group consisting of RNA, genomic DNA, synthetic DNA and cDNA.

4. An isolated nucleic acid comprising a sequence that hybridizes under stringent conditions to the nucleotide sequence of SEQ ID NO:1 or the complement of SEQ ID NO:1.

5. A purified polypeptide comprising SEQ ID NO:2.

6. An expression vector comprising the nucleic acid of claim 1, operably linked to an expression control element.

7. The expression vector of claim 6, wherein said expression control element is selected from the group consisting of constitutive, cell-specific and inducible regulatory sequences.

8. An expression vector comprising a cDNA sequence encoding a nucleotide sequence that expresses ZAC of SEQ ID NO:2.

9. A cultured cell comprising the vector of claim 6.

10. A cultured cell comprising the nucleic acid of claim 1 operably linked to an expression control element.

11. A cultured cell transformed with the vector of claim 6, wherein said cell expresses the polypeptide encoded by the nucleic acid comprising said vector.

12. The cultured cell of claim 9, wherein said cell is selected from the group consisting of eukaryotic cells and prokaryotic cells.

13. An antibody that binds specifically to ZAC.

14. The antibody of claim 13, which is a monoclonal antibody or a polyclonal antibody.

15. The antibody of claim 13, wherein said antibody prevents the activation of ZAC.

16. A therapeutic method for modulating ZAC signaling activity or signal transduction in a patient in need of treatment comprising administering to said patient an agonist, an antagonist or an inverse agonist of ZAC.

17. A method for identifying an agonist of ZAC comprising:
i) contacting a potential agonist with a cell expressing ZAC; and
ii) determining whether in the presence of said potential agonist the cell current is increased relative to the cell current in the absence of said potential agonist.

18. A method for identifying an inverse agonist to ZAC comprising:
i) contacting a potential inverse agonist with a cell expressing ZAC; and
ii) determining whether in the presence of said potential inverse agonist, the cell current is decreased relative to the cell current in the absence of said potential inverse agonist, and in the absence of an agonist.

19. The method of claim 18, wherein said agonist is zinc.

20. A method for identifying an antagonist of ZAC comprising:
iii) contacting a potential antagonist with a cell expressing ZAC; and
iv) determining whether in the presence of said potential antagonist the cell current is decreased relative to the cell current in the presence of a modulator or an agonist.

21. The method of claim 20, wherein said agonist is zinc.

22. A therapeutic composition comprising an agonist, an antagonist or an inverse agonist of ZAC capable of modulating ZAC signaling activity or transduction, and a pharmaceutically acceptable carrier, excipient or diluent.

23. A method for treating a disease comprising administering to a patient in need of treatment a therapeutic composition comprising an agonist, an antagonist or an inverse agonist of ZAC capable of modulating ZAC signaling activity or transduction, and a pharmaceutically acceptable carrier, excipient or diluent.
